(19) United States
(12) Patent Application Publication
(10) Pub. No.: US 2003/0143205 A1
(43) Pub. Date: Jul. 31, 2003

(54) Alphavirus expression systems

(75) Inventors: Jason Slingsby, London (GB); Kyriacos Mitrophanous, Oxford (GB); Jonathan Rohll, San Diego, CA (US); Alison Lawrie, Oxford (GB)

Correspondence Address: FOLEY AND LARDNER, SUITE 500, 3000 K STREET NW, WASHINGTON, DC, 20007, US

(73) Assignee: Oxford Biomedica (UK) Limited

(21) Appl. No.: 10/199,467

(22) Filed: Jul. 22, 2002

Related U.S. Application Data
(60) Provisional application No. 60/306,535, filed on Jul. 20, 2001.

(51) Int. Cl. A61K 48/00; C12N 7/00; C12N 15/867

(52) U.S. Cl. 424/93.2; 435/456; 435/235.1; 435/320.1

(57) ABSTRACT

An alpha-lentivirus vector comprising at least one alphaviral component and at least one lentiviral component, wherein the lentiviral component is capable of being packaged into a lentiviral particle after introduction of said vector into a human cell.

The present invention relates to a vector.

In particular, the present invention relates to a novel system for producing lentiviral particles.

More in particular, the present invention relates to a vector that is capable of expressing a lentiviral particle that is capable of delivering a nucleotide sequence of interest (hereinafter abbreviated to “NOI”)—or even a plurality of NOIs—to a site of interest.

More in particular, the present invention relates to a vector useful in gene therapy.

Gene therapy includes any one or more of: the addition, the replacement, the deletion, the supplementation, the manipulation etc. of one or more nucleotide sequences in, for example, one or more targeted sites—such as targeted cells. If the targeted sites are targeted cells, then the cells may be part of a tissue or an organ. General teachings on gene therapy may be found in Molecular Biology (Ed Robert Meyers, Pub VCH, such as pages 556-558).

By way of further example, gene therapy also provides a means by which any one or more of: a nucleotide sequence, such as a gene, can be applied to replace or supplement a defective gene; a pathogenic gene or gene product can be eliminated; a new gene can be added in order, for example, to create a more favourable phenotype; cells can be manipulated at the molecular level to treat cancer (Schmidt-Wolf and Schmidt-Wolf, 1994, Annals of Hematology 69;273-279) or other conditions—such as immune, cardiovascular, neurological, inflammatory or infectious disorders; antigens can be manipulated and/or introduced to elicit an immune response—such as genetic vaccination.

In recent years, retroviruses have been proposed for use in gene therapy. Essentially, retroviruses are RNA viruses with a life cycle different to that of lytic viruses. In this regard, when a retrovirus infects a cell, its genome is converted to a DNA form. In other words, a retrovirus is an infectious entity that replicates through a DNA intermediate. More details on retroviral infection etc. are presented later on.

There are many retroviruses and examples include: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

A detailed list of retroviruses may be found in Coffin et al (“Retroviruses” 1997 Cold Spring Harbour Laboratory Press Eds: J M Coffin, S M Hughes, H E Varmus pp 758-763).

Details on the genomic structure of some retroviruses may be found in the art. By way of example, details on HIV may be found from the NCBI Genbank (i.e. Genome Accession No. AF033819).

All retroviruses contain three major coding domains, gag, pol, env, which code for essential virion proteins. Nevertheless, retroviruses may be broadly divided into two categories: namely, “simple” and “complex”. These categories are distinguishable by the organisation of their genomes. Simple retroviruses usually carry only this elementary information. In contrast, complex retroviruses also code for additional regulatory proteins derived from multiple spliced messages.

Retroviruses may even be further divided into seven groups. Five of these groups represent retroviruses with oncogenic potential. The remaining two groups are the lentiviruses and the spumaviruses. A review of these retroviruses is presented in “Retroviruses” (1997 Cold Spring Harbour Laboratory Press Eds: J M Coffin, S M Hughes, H E Varmus pp 1-25).

All oncogenic members except the human T-cell leukemia virus-bovine leukemia virus group (HTLV-BLV) are simple retroviruses. HTLV, BLV and the lentiviruses and spumaviruses are complex. Some of the best studied oncogenic retroviruses are Rous sarcoma virus (RSV), mouse mammary tumour virus (MMTV) and murine leukemia virus (MLV) and the human T-cell leukemia virus (HTLV).

The lentivirus group can be split even further into “primate” and “non-primate”. Examples of primate lentiviruses include the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiencey virus (FIV) and bovine immunodeficiencey virus (BIV).

A distinction between the lentivirus family and other types of retroviruses is that lentiviruses have the capability to infect both dividing and non-dividing cells (Lewis et al 1992 EMBO. J 11; 3053-3058, Lewis and Emerman 1994 J. Virol. 68: 510-516). In contrast, other retroviruses—such as MLV—are unable to infect non-dividing cells such as those that make up, for example, muscle, brain, lung and liver tissue.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular proteins. The provirus encodes the proteins and packaging machinery required to make more virus, which can leave the cell by a process sometimes called “budding”.

As already indicated, each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral gene. Encapsidation of the retroviral RNAs occurs by virtue of a psi sequence located at the 5′ end of the viral genome.

The LTRs themselves are indentical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For ease of understanding, a simple, generic diagram (not to scale) of a retroviral genome showing the elementary features of the LTRs, gag, pol and env is presented below.

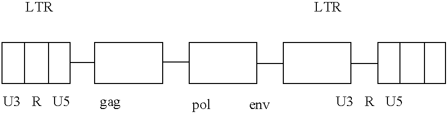

For the viral genome, the site of transcription initiation is at the boundary between U3 and R in the left hand side LTR (as shown above) and the site of poly (A) addition (termination) is at the boundary between R and U5 in the right hand side LTR (as shown above). U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

As shown in the diagram above, the basic molecular organisation of a retroviral RNA genome is (5′) R-U5-gag, pol, env-U3-R (3′). In a retroviral vector genome gag, pol and env are absent or not functional. The R regions at both ends of the RNA are repeated sequences. U5 and U3 represent sequences unique, respectively, to the 5′ and 3′ ends of the RNA genome. These three sets of end sequences go to form the long terminal repeats (LTRs) in the proviral DNA, which is the form of the genome which integrates into the genome of the infected cell. The LTRs in a wild type retrovirus consist of (5′)U3-R-U5 (3′), and thus U3 and U5 both contain sequences which are important for proviral integration. Other essential sequences required in the genome for proper functioning include a primer binding site for first strand reverse transcription, a primer binding site for second strand reverse transcription and a packaging signal.

With regard to the structural genes gag, pol and env themselves and in slightly more detail, gag encodes the internal structural protein of the virus. Gag is proteolytically processed into the mature proteins MA (matrix), CA (capsid), NC (nucleocapsid). The gene pol encodes the reverse transcriptase (RT), which contains both DNA polymerase, and associated RNase H activities and integrase (IN), which mediates replication of the genome. The gene env encodes the surface (SU) glycoprotein and the transmembrane (TM) protein of the virion, which form a complex that interacts specifically with cellular receptor proteins. This interaction leads ultimately to fusion of the viral membrane with the cell membrane.

The envelope glycoprotein complex of retroviruses includes two polypeptides: an external, glycosylated hydrophilic polypeptide (SU) and a membrane-spanning protein (TM). Together, these form an oligomeric “knob” or “knobbed spike” on the surface of a virion. Both polypeptides are encoded by the env gene and are synthesised in the form of a polyprotein precursor that is proteolytically cleaved during its transport to the cell surface. Although uncleaved Env proteins are able to bind to the receptor, the cleavage event itself is necessary to activate the fusion potential of the protein, which is necessary for entry of the virus into the host cell. Typically, both SU and TM proteins are glycosylated at multiple sites. However, in some viruses, exemplified by MLV, TM is not glycosylated.

Although the SU and TM proteins are not always required for the assembly of enveloped virion particles as such, they do play an essential role in the entry process. In this regard, the SU domain binds to a receptor molecule—often a specific receptor molecule—on the target cell. It is believed that this binding event activates the membrane fusion-inducing potential of the TM protein after which the viral and cell membranes fuse. In some viruses, notably MLV, a cleavage event—resulting in the removal of a short portion of the cytoplasmic tail of TM—is thought to play a key role in uncovering the full fusion activity of the protein (Brody et al 1994 J. Virol. 68: 4620-4627, Rein et al 1994 J. Virol. 68: 1773-1781). This cytoplasmic “tail”, distal to the membrane-spanning segment of TM remains on the internal side of the viral membrane and it varies considerably in length in different retroviruses.

Thus, the specificity of the SU/receptor interaction can define the host range and tissue tropism of a retrovirus. In some cases, this specificity may restrict the transduction potential of a recombinant retroviral vector. For this reason, many gene therapy experiments have used MLV. A particular MLV that has an envelope protein called 4070A is known as an amphotropic virus, and this can also infect human cells because its envelope protein “docks” with a phosphate transport protein that is conserved between man and mouse. This transporter is ubiquitous and so these viruses are capable of infecting many cell types. In some cases however, it may be beneficial, especially from a safety point of view, to specifically target restricted cells. To this end, several groups have engineered a mouse ecotropic retrovirus, which unlike its amphotropic relative normally only infects mouse cells, to specifically infect particular human cells. Replacement of a fragment of an envelope protein with an erythropoietin segement produced a recombinant retrovirus which then bound specifically to human cells that expressed the erythropoietin receptor on their surface, such as red blood cell precursors (Maulik and Patel 1997 “Molecular Biotechnology: Therapeutic Applications and Strategies” 1997. Wiley-Liss Inc. pp 45.).

In addition to gag, pol and env, the complex retroviruses also contain “accessory” genes which code for accessory or auxiliary proteins. Accessory or auxiliary proteins are defined as those proteins encoded by the accessory genes in addition to those encoded by the usual replicative or structural genes, gag, pol and env. These accessory proteins are distinct from those involved in the regulation of gene expression, like those encoded by tat, rev, tax and rex. Examples of accessory genes include one or more of vif vpr, vpx, vpu and nef These accessory genes can be found in, for example, HIV (see, for example pages 802 and 803 of “Retroviruses” Ed. Coffin et al Pub. CSHL 1997). Non-essential accessory proteins may function in specialised cell types, providing functions that are at least in part duplicative of a function provided by a cellular protein. Typically, the accessory genes are located between pol and env, just downstream from env including the U3 region of the LTR or overlapping portions of the env and each other.

The complex retroviruses have evolved regulatory mechanisms that employ virally encoded transcriptional activators as well as cellular transcriptional factors. These trans-acting viral proteins serve as activators of RNA transcription directed by the LTRs. The transcriptional trans-activators of the lentiviruses are encoded by the viral tat genes. Tat binds to a stable, stem-loop, RNA secondary structure, referred to as TAR, one function of which is to apparently optimally position Tat to trans-activate transcription.

As mentioned earlier, retroviruses have been proposed as a delivery system (other wise expressed as a delivery vehicle or delivery vector) for inter alia the transfer of a NOI, or a plurality of NOIs, to one or more sites of interest. The transfer can occur in vitro, ex viva, in vivo, or combinations thereof. When used in this fashion, the retroviruses are typically called retroviral vectors or recombinant retroviral vectors. Retroviral vectors have even been exploited to study various aspects of the retrovirus life cycle, including receptor usage, reverse transcription and RNA packaging (reviewed by Miller, 1992 Curr Top Microbiol Immunol 158:1-24).

In a typical recombinant retroviral vector for use in gene therapy, at least part of one or more of the gag, pol and env protein coding regions may be removed from the virus. This makes the retroviral vector replication-defective. The removed portions may even be replaced by a NOI in order to generate a virus capable of integrating its genome into a host genome but wherein the modified viral genome is unable to propagate itself due to a lack of structural proteins. When integrated in the host genome, expression of the NOI occurs—resulting in, for example, a therapeutic effect. Thus, the transfer of a NOI into a site of interest is typically achieved by: integrating the NOI into the recombinant viral vector; packaging the modified viral vector into a virion coat; and allowing transduction of a site of interest—such as a targeted cell or a targeted cell population.

It is possible to propagate and isolate quantities of retroviral vectors (e.g. to prepare suitable titres of the retroviral vector) for subsequent transduction of, for example, a site of interest by using a combination of a packaging or helper cell line and a recombinant vector.

In some instances, propagation and isolation may entail isolation of the retroviral gag, pol and env genes and their separate introduction into a host cell to produce a “packaging cell line”. The packaging cell line produces the proteins required for packaging retroviral DNA but it cannot bring about encapsidation due to the lack of a psi region. However, when a recombinant vector carrying a NOI and a psi region is introduced into the packaging cell line, the helper proteins can package the psi-positive recombinant vector to produce the recombinant virus stock. This can be used to infect cells to introduce the NOI into the genome of the cells. The recombinant virus whose genome lacks all genes required to make viral proteins can infect only once and cannot propagate. Hence, the NOI is introduced into the host cell genome without the generation of potentially harmful retrovirus. A summary of the available packaging lines is presented in “Retroviruses” (1997 Cold Spring Harbour Laboratory Press Eds: J M Coffin, S M Hughes, H E Varmus pp 449). However, this technique can be problematic in the sense that the titre levels are not always at a satisfactory level. Nevertheless, the design of retroviral packaging cell lines has evolved to address the problem of inter alia the spontaneous production of helper virus that was frequently encountered with early designs. As recombination is greatly facilitated by homology, reducing or eliminating homology between the genomes of the vector and the helper has reduced the problem of helper virus production.

More recently, packaging cells have been developed in which the gag, pol and env viral coding regions are carried on separate expression plasmids that are independently transfected into a packaging cell line so that three recombinant events are required for wild type viral production. This strategy is sometimes referred to as the three plasmid transfection method (Soneoka et al 1995 Nucl. Acids Res. 23: 628-633).

Transient transfection can also be used to measure vector production when vectors are being developed. In this regard, transient transfection avoids the longer time required to generate stable vector-producing cell lines and is used if the vector or retroviral packaging components are toxic to cells. Components typically used to generate retroviral vectors include a plasmid encoding the Gag/Pol proteins, a plasmid encoding the Env protein and a plasmid containing a NOI. Vector production involves transient transfection of one or more of these components into cells containing the other required components. If the vector encodes toxic genes or genes that interfere with the replication of the host cell, such as inhibitors of the cell cycle or genes that induce apotosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al 1993, PNAS 90:8392-8396).

In view of the toxicity of some HIV proteins—which can make it difficult to generate stable HIV-based packaging cells—HIV vectors are usually made by transient transfection of vector and helper virus. Some workers have even replaced the HIV Env protein with that of vesicular stomatis virus (VSV). Insertion of the Env protein of VSV facilitates vector concentration as HIV/VSV-G vectors with titres of 5×10⁵ (10⁸ after concentration) were generated by transient transfection (Naldini et al 1996 Science 272: 263-267). Thus, transient transfection of HIV vectors may provide a useful strategy for the generation of high titre vectors (Yee et al 1994 PNAS. 91: 9564-9568). A drawback, however, with this approach is that the VSV-G protein is quite toxic to cells.

Thus, and as indicated, retroviral vectors are used extensively in biomedical research and for gene therapy. Current methods for the production of retroviral vectors make use of the fact that the two roles of the wild-type retrovirus genome, that is protein encoding and as a template for new genome copies, can be de-coupled (e.g. Soneoka et al 1995 Nucl. Acids Res. 23, 628 and references therein). Protein that is required for the assembly of new virus particles and for enzyme and regulatory functions can be produced by non-genome sequences in, for example, a mammalian packaging cell line (e.g. Miller 1990 Hum. Gene Therapy 1, 5). A genome sequence lacking the protein encoding functions is provided, so that the resulting retroviral vector particles are capable of infecting but not of replicating in a target cell. The genome sequence can also be designed for delivery and integration of a therapeutic gene (Vile and Russel 1995 Brit. Med. Bull 51, 12). Standard methods for producing murine leukaemia virus (MLV)-based vectors, for example, include use of cell lines expressing the gag-pol and env genes (the packaging components) of MLV. These will package a compatible retroviral vector genome introduced by transduction or by transfection with an appropriate plasmid. An alternative method involves simultaneous transfection of gag-pol, env, and vector genome plasmids into suitable cells.

Although the principles of these systems are well understood, in practice the re-constructed virus assembly system often fails to generate the quantity of vector particles which will be required in practice for use in gene therapy. Retroviral vector particles are generally harvested by removing supernatant from a culture of particle-producing cells. The resulting suspension may be concentrated with respect to the vector particles, using physical methods, but only to a limited degree as problems such as aggregation and damage tend to arise. Thus, it may only be possible to concentrate a suspension of vector particles by up to 100-fold.

The present invention seeks to provide a new and improved approach to the production of lentiviruses.

### SUMMARY OF THE INVENTION

Semliki forest virus (SFV) based vectors have been previously used for the generation of recombinant Moloney Murine Leukaemia virus vectors (MLV) (Garoff-WO98/15636). However, important clinical targets for gene therapy include terminally differentiated or slowly dividing cells such as haematopoetic stem cells, those found in the CNS or in solid tumours. Gene transfer to these tissues is inefficient using MLV-based vectors due to their inability to transduce non dividing cells. As discussed above lentiviruses have the ability to infect mitotically active and inactive cells. One limitation with lentiviral production systems to-date has been the transduction of genes containing introns which are usually removed from the RNA by the nuclear splicing machinery. As the presence of introns has been shown to be important for maximising the expression of many therapeutic gene cassettes, cytoplasmic production as provided for by SFV processing would allow their retention. Thus, cytoplasmically produced lentiviruses will allow the inclusion of introns and enhancer elements in the delivery of therapeutic genes cassettes.

Garoff only specifically exemplifies retroviral production in BHK cells. Garoff also discusses “we have in our examples only shown how to produce MLV vectors using alphavirus vectors but it should be equally possible to use our system for the production of other retrovirus based vectors, e.g. HIV-1, vectors”. Garoff goes on to specifically exemplifies retroviral production in BHK cells. However, surprisingly and quite contrary to the teaching of Garoff we have found that BHK cells are not permissive for lentiviral replication showing that in these cells there is a block in gag processing and budding. We have now surprisingly found that it is necessary to use human cells.

Surprising Features/Advantages

We have found that human cells, such as 293T cells support lentiviral, such as EIAV, replication and do not exhibit blocks in viral assembly and budding. BHK cells on the other hand do show definite blocks in Gag processing and assembly and are therefore non-permissive for lentiviral, as exemplified by EIAV, replication.

Statements of the Invention

The present invention seeks to provide an improved system for preparing viral particles that may be of subsequent use in medicine.

In particular, the present invention seeks to provide an improved system for preparing a high titre of viral particles that may be of subsequent use in medicine.

According to a first aspect of the present invention there is provided an alpha-lentivirus RNA vector comprising at least one alphaviral component and at least one lentiviral component, wherein the lentiviral component is capable of being packaged into a lentiviral particle after introduction of said vector into a human cell.

According to a second aspect of the present invention there is provided a vector wherein the vector is a alphavirus expression system comprising at least one lentiviral component, wherein the lentiviral component is capable of being packaged into a lentiviral particle after introduction of said vector into a human cell.

According to a third aspect of the present invention there is provided a lentiviral particle obtainable from expression of the vector according to the present invention.

According to a fourth aspect of the present invention there is provided a process for preparing a lentiviral particle comprising expressing the vector according to the present invention.

According to a fifth aspect of the present invention there is provided a human cell comprising the vector according to the present invention.

According to a sixth aspect of the present invention there is provided a lentiviral vector particle production system comprising the vector according to the present invention in a human cell.

According to a seventh aspect of the present invention there is provided a lentiviral vector particle produced by the lentiviral vector particle production system according to the present invention.

According to an eighth aspect of the present invention there is provided an expression vector comprising a polynucleotide sequence which encodes a lentiviral vector genome having a 5′ and a 3′ end, which lentiviral vector genome is capable of being expressed and packaged into a lentiviral vector particle in a alphavirus expression system.

Preferably the lentiviral component corresponds to a lentiviral genome.

Preferably the composition comprises an RNA transcription start site for the lentiviral vector genome, and wherein the nucleotide sequence encoding the lentiviral component is operably linked to a promoter comprising an upstream promoter component located upstream of the RNA transcription start site and a downstream promoter component located downstream of the RNA transcription start site.

Preferably the downstream promoter component is upstream of the polynucleotide sequence encoding the lentiviral vector genome.

In one embodiment, preferably the promoter is a alphavirus promoter.

In one embodiment, preferably the promoter is a non-alphavirus promoter.

Preferably the promoter is the T7 promoter or the sp6 Salmonella phage promoter.

Preferably the composition comprises at least one RNA cleavage component.

Preferably manipulation of at least one of the RNA cleavage component(s) could yield a retroviral genome free of any lentiviral components.

Preferably at least one of the RNA cleavage component(s) is located between the promoter and the sequence encoding the lentiviral component.

Preferably at least one of the RNA cleavage component(s) is located immediately adjacent the sequence encoding the lentiviral vector component for subsequent cleavage at the 5′ end of the vector component.

Preferably at least one of the RNA cleavage component(s) is located downstream of the sequence encoding the lentiviral component.

Preferably the RNA cleavage component(s) has a cleavage site immediately adjacent the sequence encoding the lentiviral vector component for subsequent cleavage at the 3′ end of the vector component.

Preferably at least one of the RNA cleavage component(s) is a ribozyme cleavage site for subsequent cleavage thereof.

Preferably each RNA cleavage component is a ribozyme cleavage site for subsequent cleavage thereof.

The or each ribozyme cleavage site may be cleaved by a ribozyme which is independently derived from the composition. Preferably, however, any one or more of the ribozyme cleavage sites is cleaved by a ribozyme derived from the second viral component.

Ribozymes are RNA molecules that can function to catalyse specific chemical reactions within cells without the obligatory participation of proteins. For example, group I ribozymes take the form of introns which can mediate their own excision from self-splicing precursor RNA. Other ribozymes are derived from self-cleaving RNA structures which are essential for the replication of viral RNA molecules. Like protein enzymes, ribozymes can fold into secondary and tertiary structures that provide specific binding sites for substrates as well as cofactors, such as metal ions. Examples of such structures include hammerhead, hairpin or stem-loop, pseudoknot and hepatitis delta antigenomic ribozymes have been described.

Each individual ribozyme has a motif which recognises and binds to a recognition site in a target RNA. This motif takes the form of one or more “binding arms” but generally two binding arms. The binding arms in hammerhead ribozymes are the flanking sequences Helix I and Helix III which flank Helix II. These can be of variable length, usually between 6 to 10 nucleotides each, but can be shorter or longer. The length of the flanking sequences can affect the rate of cleavage. For example, it has been found that reducing the total number of nucleotides in the flanking sequences from 20 to 12 can increase the turnover rate of the ribozyme cleaving a HIV sequence, by 10-fold (Goodchild, JVK, 1991 Arch Biochem Biophys 284: 386-391). A catalytic motif in the ribozyme Helix II in hammerhead ribozymes cleaves the target RNA at a site which is referred to as the cleavage site. Whether or not a ribozyme will cleave any given RNA is determined by the presence or absence of a recognition site for the ribozyme containing an appropriate cleavage site.

Each type of ribozyme recognizes its own cleavage site. The hammerhead ribozyme cleavage site has the nucleotide base triplet GUX directly upstream where G is guanine, U is uracil and X is any nucleotide base. Hairpin ribozymes have a cleavage site of BCUGNYR, where B is any nucleotide base other than adenine, N is any nucleotide, Y is cytosine or thymine and R is guanine or adenine. Cleavage by hairpin ribozymes takes places between the G and the N in the cleavage site.

More details on ribozymes may be found in “Molecular Biology and Biotechnology” (Ed. R A Meyers 1995 VCH Publishers Inc p831-8320 and in “Retroviruses” (Ed. J M Coffin et al 1997 Cold Spring Harbour Laboratory Press pp 683).

In a particularly preferred embodiment added to the 5′ end of the lentiviral component is both the 3′ region of the alphavirus genome promoter and the coding region of nonstructural protein 4, and added to the 3′ end of the lentiviral component is a viral RNA replication signal (Strauss & Strauss (1994) Microbiol. Rev. 58:491-562).

The sequences required for efficient transcription from the alphavirus subgenomic promoter may extend into the transcript itself. For expression of a protein from a transcript made from this promoter, for example the EIAV gag-pol placed downstream of the SFV promoter, this characteristic is not a problem. However, for production of a transcript representing a lentiviral genome it may cause a difficulty. This arises because the lentiviral vector transcript will have a 5′ extension derived from alphavirus which after the “first strand jump” step of reverse transcription will not have a complementary sequence (within the 3′ LTR) to which to anneal. This lack of complementarity causes inefficient negative strand DNA synthesis and poor vector titre. The solution is to alter the 3′ LTR to match the 5′ end of the transcript. This can be achieved by, e.g. insertion of alphavirus sequence upstream of the R region of the lentivirus 3′ LTR.

Alphaviruses

Alphaviruses are among a large group of viruses whose members were originally classified as arboviruses because they replicate in both vertebrate and invertebrate cells. Vertebrates and mammals are usually infected through the bite of an invertebrate, such as a mosquito. Several alphaviruses, including Venezuelan equine encephalitis (VEE) virus and Ross River virus, are known human pathogens, but attenuated variants exist. Two other alphaviruses, Sindbis virus and Semliki Forest virus, are better known as models used for a variety of studies in molecular and cell biology.

The alphavirus genomic RNA is approximately 12×10³ nucleotides; it is capped at the 5′ terminus and polyadenylated at the 3′ terminus. The 5′ two-thirds of the genome contains the genetic information for the nonstructural proteins (nsPs), which are required for transcription and replication of the RNA. The 3′ one-third of the genome contains genes encoding the viral structural proteins—the capsid protein, a hydrophobic 6K protein, and two viral envelope glycoproteins.

FIG. 4 depicts the steps in replication, transcription and translation that occur once the viral RNA enters the cytoplasm of the infected cell. Only the nonstructural proteins (nsPs) are directly translated. They are encoded in a single open reading frame, which is translated as a polyprotein that is co- and posttranslationally cleaved to form four polypeptides. They function together in a replication complex that is required for the synthesis of the complementary negative strand. It serves as a template not only for the synthesis of more virion RNA, but also for the transcription of a subgenomic RNA that is identical in sequence to the 3′ one-third of the genome.

The nucleotides spanning the junction between the nsPs and the structural protein genes on the negative strand serve as the promoter for transcription of the subgenomic RNA. The viral structural proteins are translated from this latter RNA. They are also synthesized as a polyprotein with the amino-terminal capsid protein functioning as an autoprotease to cleave itself from the nascent polypeptide. The capsid protein can then interact with the genomic RNA forming the nucleocapsid.

There are specific recognition signals located within the nsP coding sequence which serve as a nucleation site for encapsidation and ensure that the genomic RNA, but not the subgenomic RNA, is packaged. The division into separate modules is the basis for most of the expression vectors derived from the alphavirus genome.

As shown in FIG. 4, in the cytoplasm of the infected cell, the 5′ ⅔s of the alphavirus genome is translated from a single site of initiation to yield 4 proteins (step 1). These proteins are required for the synthesis of the complementary (−) strand (step 2) which is the template for replication of genomic RNA and transcription of subgenomic RNA (step 3). The subgenomic RNA is translated to produce the structural proteins (step 4) which interact with the genomic RNA and assemble into virus particles (step 5). The horizontal arrow shown on the complementary ([minus]) strand indicates the subgenomic RNA promoter.

Alphavirus Expression Vectors

All four of the better-known alphaviruses—Sindbis virus, Semliki Forest virus, VEE virus, and Ross River virus—are cytopathic to vertebrate cells in culture. Moreover, they all inhibit host cell protein synthesis, permitting viral subgenomic RNA to be translated very efficiently and the viral structural proteins to become the dominant proteins in the cell.

One of the initial goals in developing alphaviruses as expression vectors was to subvert the alphavirus subgenomic RNA to synthesize heterologous proteins instead of the viral structural proteins. The first example of a self-replicating alphavirus expression vector was a modified Sindbis virus in which the structural protein genes were replaced by the bacterial chloramphenicol acetyltransferase gene. The ability of this modified viral RNA to replicate and produce a foreign protein established the concept that an alphavirus can express a heterologous protein. It also proved that alphavirus RNA transcription and replication do not depend on the presence of the structural protein genes or their protein products.

Now several different types of alphavirus vectors can be used for protein expression. The first version, designated as replicons, is self-replicating. Replicons are introduced into cultured cells by transfection. For instance, when used in baby hamster kidney (BHK) cells, transfection can be very efficient-sometimes, nearly 100% of the cells are transfected by electroporation.

These replicons also can be introduced as cDNA plasmids under the control of a eukaryotic promoter. Expression from a DNA plasmid has proved to be an effective means for introducing replicons into cells both in culture and in animals. Alphavirus replicons have been termed “suicide vectors” because they do not spread beyond the initial cell they enter.

One advantage of using viruses to introduce genes into cells is that they are effective as delivery systems. Moreover, infection is usually more efficient and less stressful to the cell than transfection. Replicons can be packaged into extracellular particles when they are cotransfected into cells with defective helper nucleic acid to provide the structural proteins. Defective helpers lack most of the nsP genes and are not self-replicating. They contain the genes for the structural proteins and will translate them from a subgenomic RNA when the latter is transcribed by the nsPs that are translated from replicon genomes.

Any alphavirus may be used in the present invention and these include Sindbis virus, Semliki Forest virus (SFV), Ross River virus, Venezuelan Equine Encephalitis virus (VEE), Western and Easter Equine Encephalitis virus. SFV is particularly preferred.

Packaging Cell

As used herein, the term “packaging cell” refers to a cell which contains those elements necessary for production of infectious recombinant virus which are lacking in a recombinant viral vector. Typically, such packaging cells contain one or more expression cassettes which are capable of expressing viral structural proteins (such as gag, pol and env) but they do not contain a packaging signal.

In preferred packaging and producer cells, the toxic envelope protein sequences, and nucleocapsid sequences are all stably integrated in the cell. However, one or more of these sequences could also exist in episomal form and gene expression could occur from the episome.

Packaging cell lines may be readily prepared (see also WO 92/05266), and utilised to create producer cell lines for the production of lentiviral vector particles. A summary of the available packaging lines is presented in “Retroviruses” (see below). Packaging cell lines suitable for use with the present invention are readily prepared (see also WO 92/05266).

Simple packaging cell lines, comprising a provirus in which the packaging signal has been deleted, have been found to lead to the rapid production of undesirable replication competent viruses through recombination. In order to improve safety, second generation cell lines have been produced wherein the 3′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

Preferably, the packaging cell lines are second generation packaging cell lines.

Preferably, the packaging cell lines are third generation packaging cell lines.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production.

The packaging cell line is a human cell line, such as for example: HEK 293, HEK 293T, HEK 293 101, TE671, HT1080.

Preferably the packaging cell is derived from a HEK 293T cell. Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

The packaging cell lines of the present invention provide the gene products necessary to encapsidate and provide a membrane protein for a viral vehicle such as a retrovirus and retrovirus nucleic gene delivery vehicle. As described below, when viral sequences such as lentivirus sequences are introduced into the packaging cell lines, such sequences are encapsidated with the nucleocapsid proteins and these units then bud through the cell membrane to become surrounded in cell membrane and to contain the envelope protein produced in the packaging cell line. These infectious lentiviruses are useful as infectious units per se or as gene delivery vectors

Producer Cell

As used herein, the term “producer cell” or “vector producing cell” refers to a cell which contains all the elements necessary for production of a lentiviral vector such as recombinant lentiviral vectors, recombinant lentiviral vector particles and lentiviral delivery systems.

The process of making a producer cell for a lentiviral vector involves establishment of cell lines that express the components required for lentivector particle production (for example gag/pol, vector genome and envelope). In such cell lines, viral sequences such as lentiviral sequences are capable of being packaged with the nucleocapsid proteins. Further, the viral sequences such as lentiviral sequences that are capable of being packaged may also contain one or more heterologous nucleotide of interest (NOI) that are capable of being expressed in a target cell that is infected by the virions produced in the producer cell.

Preferably, the producer cell is obtainable from a stable producer cell line.

The producer cell lines of the present invention as utilised for the production of infectious pseudotyped lentivirus, and vector particles and especially high titer virions which may also contain one or more NOIs capable of being expressed in a target cell or tissue. The cells are thus useful for packaging a lentiviral vector genome which may also contain a heterologous NOI capable of being expressed in a target cell or tissue.

There are two common procedures for generating viral producer cells such as lentiviral producer cells. In one, the sequences encoding lentiviral Gag, Pol and Env proteins are introduced into the cell and stably integrated into the cell genome; a stable cell line is produced which is referred to as the packaging cell line. The packaging cell line produces the proteins required for packaging lentiviral RNA but it cannot bring about encapsidation due to the lack of apsi region. However, when a vector genome (having a psi region) is introduced into the packaging cell line, the helper proteins can package the psi-positive recombinant vector RNA to produce the recombinant vector stock. This can be used to transduce the NOI into recipient cells. The recombinant virus whose genome lacks all genes required to make viral proteins can infect only once and cannot propagate. Hence, the NOI is introduced into the host cell genome without the generation of potentially harmful lentivirus. As already mentioned above, a summary of the available packaging lines is presented in “Retroviruses” (1997 Cold Spring Harbour Laboratory Press Eds: J M Coffin, S M Hughes, H E Varmus pp 449).

The second approach is to introduce the three different DNA sequences that are required to produce a lentiviral vector particle i.e. the env coding sequences, the gag-pol coding sequence and the defective lentiviral genome containing one or more NOIs into the cell at the same time by transient transfection and the procedure is referred to as transient triple transfection (Landau & Littman 1992; Pear et al 1993). The triple transfection procedure has been optimised (Soneoka et al 1995; Finer et al 1994). WO 94/29438 describes the production of producer cells in vitro using this multiple DNA transient transfection method. WO 97/27310 describes a set of DNA sequences for creating retroviral producer cells either in vivo or in vitro for re-implantation.

The components of the viral system which are required to complement the vector genome may be present on one or more “producer plasmids” for transfecting into cells.

The present invention also provides a kit for producing an alpha-lentiviral vector system, comprising

(i) an alphavirus vector comprising a lentiviral vector genome which is incapable of encoding one or more proteins which are required to produce a lentivector particle;

(ii) one or more producer plasmid(s) capable of encoding the protein which is not encoded by (i); and optionally

(iii) a human cell suitable for conversion into a producer cell.

In a preferred embodiment, the lentiviral vector genome is incapable of encoding the proteins gag, pol and env. Preferably the kit comprises one or more producer plasmids encoding env, gag and pol, for example, one producer plasmid encoding env and one encoding gag-pol. Preferably the gag-pol sequence is codon optimised for use in the particular producer cell (see below).

The present invention also provides a producer cell expressing the lentivector genome and the producer plasmid(s) capable of producing a lentiviral vector system useful in the present invention.

By using producer/packaging cell lines, it is possible to propagate and isolate quantities of lentiviral vector particles (e.g. to prepare suitable titres of the lentiviral vector particles) for subsequent transduction of, for example, a site of interest (such as adult brain tissue). Producer cell lines are usually better for large scale production or vector particles.

Transient transfection has some advantages over the packaging cell method. In this regard, transient transfection avoids the longer time required to generate stable vector-producing cell lines and is used if the vector genome or lentiviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al 1993, PNAS 90:8392.8396). However, the transient method for production of vector has the disadvantage that it is labour intensive, it is not readily adapted to production of vector on a large scale and it is difficult to ensure uniformity between vectors produced by the method at different times.

Producer cells/packaging cells can be of any suitable human cell type.

As used herein, the term “producer cell” or “vector producing cell” refers to a cell which contains all the elements necessary for production of lentiviral vector particles.

Preferably, the producer cell is obtainable from a stable producer cell line.

Preferably, the producer cell is obtainable from a derived stable producer cell line.

Preferably, the producer cell is obtainable from a derived producer cell line.

As used herein, the term “derived producer cell line” is a transduced producer cell line which has been screened and selected for high expression of a marker gene. Such cell lines support high level expression from the lentiviral genome. The term “derived producer cell line” is used interchangeably with the term “derived stable producer cell line” and the term “stable producer cell line.

Preferably the derived producer cell line includes but is not limited to a lentiviral producer cell.

Preferably the derived producer cell line is an HIV or EIAV producer cell line, more preferably an EIAV producer cell line.

Preferably the envelope protein sequences, and nucleocapsid sequences are all stably integrated in the producer and/or packaging cell. However, one or more of these sequences could also exist in episomal form and gene expression could occur from the episome.

Vector

As it is well known in the art, a vector is a tool that allows or faciliates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the nucleotide sequences (NS) of the present invention and/or expressing the proteins of the invention encoded by the nucleotide sequences (NS) of the present invention. Examples of vectors used in recombinant DNA techniques include but are not limited to plasmids, chromosomes, artificial chromosomes or viruses.

The term “vector” includes expression vectors and/or transformation vectors.

The term “transformation vector” means a construct capable of being transferred from one species to another.

The term “expression vector” means a construct capable of in vivo or in vitrolex vivo expression.

Expression Vector

Preferably, a nucleotide sequence (NS) of present invention which is inserted into a vector is operably linked to a control sequence that is capable of providing for the expression of the coding sequence by the host cell, i.e. the vector is an expression vector. The NS produced by a host recombinant cell may be secreted or may be contained intracellularly depending on the sequence and/or the vector used.

Nucleotide Sequence (NS)

The NS of the present invention refers to the lentiviral component of the alpha-lentivirus vector of the present invention and/or the NOI which may be present in the lentiviral component.

Expression Vector

The term “expression vector” as used in the present invention refers to an assembly which is capable of directing the expression of a nucleotide sequence. The NS expression vector must include a promoter which, when transcribed, is operably linked to the NS, as well as a polyadenylation sequence. Within other embodiments of the invention, the expression vectors described herein may be contained within a plasmid construct.

Expression Cassette

The term “expression cassette” refers to a recombinant DNA molecule containing a desired coding sequence and appropriate nucleic acid sequences necessary for the expression of the operably linked coding sequence in a particular host cell such as a packaging cell. Nucleic acid sequences necessary for expression in eucaryotic cells usually include promoters, enhancers, and termination and polyadenylation signals. The cassette can be removed and inserted into a vector or plasmid as a single unit.

Expression in vitro

The vectors of the present invention may be transformed or transfected into a suitable host cell (such as a human packaging/producer cell) as described below to provide for expression of an NS. This process may comprise culturing a host cell and/or target cell transformed with an expression vector under conditions to provide for expression by the vector of an NS and optionally recovering the expressed NS. The vectors may contain one or more selectable marker genes, for example an ampicillin resistance gene in the case of a bacterial plasmid or a neomycin resistance gene for a mammalian vector. The expression of the NOI may be constitutive such that they are continually produced, or inducible, requiring a stimulus to initiate expression. In the case of inducible expression, the production of the NOI may be initiated when required by, for example, addition of an inducer substance to the culture medium, for example tetracycline or a functional analogue thereof.

Non-Viral Delivery

Alternatively, the vectors comprising nucleotide sequences (NS) of the present invention may be introduced into suitable host cells, such as packaging cells, using a variety of non-viral techniques known in the art, such as transfection, transformation, electroporation and biolistic transformation.

As used herein, the term “transfection” refers to a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationiC enzyme-mediated, cationic facial amphiphiles (CFAs) (Nature Biotechnology 1996 14; 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16: 421) and combinations thereof.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Viral Vectors

The producer cells of the present invention are used to produce lentiviral vectors.

Preferably the vector is a recombinant lentiviral vectors.

Viral Particles

In the present invention, several terms are used interchangeably. Thus, “virion”, “virus”, “viral particle”, “viral vector”, and “vector particle” mean virus and virus-like particles that are capable of introducing a nucleic acid into a cell through a viral-like entry mechanism. Such vector particles can, under certain circumstances, mediate the transfer of NOIs into the cells they infect. By way of example, a lentivirus is capable of reverse transcribing its genetic material into DNA and incorporating this genetic material into a target cell's DNA upon transduction. Such cells are designated herein as “target cells”.

Lentiviral Vectors

In the present invention, lentiviral vectors are produced.

Lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et al1992 EMBO. J 11: 3053-3058; Lewis and Emerman 1994 J. Virol. 68: 510-516). In contrast, other retroviruses—such as MLV—are unable to infect non-dividing cells such as those that make up a large proportion of, for example, muscle, brain, lung and liver tissue.

Preferred vectors for use in accordance with the present invention are recombinant lentiviral vectors, in particular recombinant lentiviral vectors, in particular minimal lentiviral vectors, teachings relating to which are disclosed in WO 99/32646 and in WO98/17815.

Preferably the recombinant lentiviral vector (RLV) of the present invention has a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell.

Minimal Systems

It has been demonstrated that a primate lentivirus minimal system can be constructed which requires none of the HIV/SIV additional genes vif vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815.

Thus, preferably, the delivery system used in the invention is devoid of at least tat and S2 (if it is an EIAV vector system), and possibly also vif vpr, vpx, vpu and nef More preferably, the systems of the present invention are also devoid of rev. Rev was previously thought to be essential in some retroviral genomes for efficient virus production. For example, in the case of HIV, it was thought that rev and RRE sequence should be included. However, it has been found that the requirement for rev and RRE can be reduced or eliminated by codon-optimisation (see below) or by replacement with other functional equivalent systems such as the Mason-Pfizer monkey virus (MPMV) constitutive transport element (CTE) system. As expression of the codon optimised gag-pol is REV independent, RRE can be removed from the gag-pol expression cassette, thus removing any potential for recombination with any RRE contained on the vector genome.

In a preferred embodiment, the viral genome of the present invention lacks the Rev response element (RRE).

In another preferred embodiment, the viral genome of the present invention comprises a polynucleotide response element.

Preferably the lentiviral genome of the present invention comprises a polynucleotide response element.

Preferably the polynucleotide response element is an RRE.

Preferably the polynucleotide response element is responsive to a nucleus to cytoplasm transport factor.

Preferably the polynucleotide response element is a wood chuck hepatitis virus post-transcriptional regulatory element (WHV PRE).

In a preferred embodiment, the system used in the present invention is based on a so-called “minimal” system in which some or all of the additional genes have be removed.

A minimal lentiviral genome for use in the present invention will therefore comprise (5′) R-U5-one or more first nucleotide sequences-U3-R (3′). However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed lentiviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included. However the requirement for rev and RRE may be reduced or eliminated by codon optimisation.

Codon Optimisation

As used herein, the terms “codon optimised” and “codon optimisation” refer to an improvement in codon usage. By way of example, alterations to the coding sequences for viral components may improve the levels of expression of those sequences in the cells which are to act as the producer cells for lentiviral vector particle production. This is referred to as “codon optimisation”. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has previously been described in WO99/41397. Different cells differ it their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corrsponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles, for example lentiviral particles, required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised.

Codon optimisation also overcomes the Rev/RRE requirement for nuclear-cytoplasmic export of lentiviral gag/pol mRNA, rendering expression from the codon-optimised sequences Rev-independent. Codon optimisation also reduces the potential for homologous recombination between different constructs within the vector system (for example between the regions of overlap in the vector, gag-pol and env open reading frames). The overall effect of codon optimisation is therefore a notable increase in viral titre and improved safety.

The gag-pol sequences of the present invention are codon optimised in their entirety, with the exception of the sequence encompassing the frameshift site.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the—gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is not codon optimised. Retaining this fragment enables more efficient expression of the gag-pol proteins.

For EIAV the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation may be based on highly expressed mammalian genes. The third and sometimes the second and third base may be changed.

Due to the degenerate nature of the Genetic Code, it will be appreciated that numerous gag-pol sequences may be achieved by a skilled worker. Also there are many lentiviral variants described which can be used as a starting point for generating a codon optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at http://hiv-web.lan1.gov. Details of EIAV clones may be found at the NCBI database: http:www.ncbi.n1m.nih.gov.

The strategy for codon optimised gag-pol sequences can be used in relation to any retrovirus. This would apply to all lentiviruses, including EIAV, FIV, BIV, CAEV, VMR, SIV, HIV-1 and HIV-2.

In one embodiment, the vector of the present invention is produced using a codon-optimised gag-pol in the vector production system.

A codon-optimised gag/pol gene is advantageous because codon-optimised gag/pol gene is packaged significantly less efficiently than the wild type gene and represents a significant improvement to the safety profile of the system.

An additional benefit that results from creation of a ‘synthetic’ codon-optimised gag/pol is that its expression becomes independent of REV/RRE. Therefore if expression of the vector component RNA can be made REV/RRE independent the potential problems of supplying REV at a sufficient level within a packaging cell to allow efficient production of infectious vector particles can be avoided. Thus, the use of a codon-optimised gag/pol gene is advantageous in cells, such as TE671 or 293 cells, where high levels of REV expression can not be tolerated,

As described above, the packaging components for a lentiviral vector include expression products of gag, pol and env genes. In addition, efficient packaging may depend on a short sequence of 4 stem loops followed by a partial sequence from gag and env (the “packaging signal”). Thus, inclusion of a deleted gag sequence in the lentiviral vector genome (in addition to the full gag sequence on the packaging construct) may optimise vector titre. To date efficient packaging has been reported to require from 255 to 360 nucleotides of gag in vectors that still retain env sequences, or about 40 nucleotides of gag in a particular combination of splice donor mutation, gag and env deletions. The present invention demonstrates the surprising finding that a deletion of all but the N-terminal 360 or so nucleotides in gag leads to an increase in vector titre. Thus, preferably, the lentiviral vector genome includes a gag sequence which comprises one or more deletions, more preferably the gag sequence comprises about 360 nucleotides derivable from the N-terminus.

Self-Inactivating (SIN) Vector

In one embodiment of the present invention, the viral vector is a self-inactivating vector.

By way of example, self-inactivating lentiviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al 1986 Proc Natl Acad Sci 83: 3194-3198; Dougherty and Temin 1987 Proc Natl Acad Sci 84: 1197-1201; Hawley et al 1987 Proc Natl Acad Sci 84: 2406-2410; Yee et al 1987 Proc Natl Acad Sci 91: 9564-9568). However, any promoter(s) internal to the LTRs in such vectors will still be transcriptionally active.

This strategy has been employed to eliminate effects of the enhancers and promoters in the viral LTRs on transcription from internally placed genes. Such effects include increased transcription (Jolly et al 1983 Nucleic Acids Res 11: 1855-1872) or suppression of transcription (Emerman and Temin 1984 Cell 39: 449-467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

Although a SIN vector may improve biosafety, the self-inactivating feature of some SIN vectors precludes vector transduction of packaging cell lines as a method of generating stable SIN vector-producing lines. Although stable SIN vector-producing cell lines have been generated by co-transfecting vector DNA with a selection marker gene into packaging cells and screening for stable cell clones yielding the highest vector titres, this approach has the shortcoming that most of the positively screening clones are genetically unstable and often subject to transcription shut off.

Recently, another approach has been the generation of stable SIN lentivirus vector producer cell lines by transduction using a conditional SIN (cSIN) vector. This approach allows efficient transcription and packaging of full length vector RNA in vector-transduced packaging cells only, yet retains its self-inactivating properties when infecting normal target cells. In this regard, Xu et al (Mol Therapy 2001 3 (1): 97-104) have developed a new vector design using a tetracycline-inducible system in which the 3′ LTR U3 transcription regulatory elements (including the TATA box) with the Tetracycline responsive element (TRE) which contains seven copies of the 42 bp tet operator sequence. Consequently, after transduction, transcription of full-length vector RNA becomes dependent on the presence of the synthetic tetracycline-regulated transactivator (tTA). The Xu et al study demonstrates that a stable SIN lentivirus vector producer line can be produced using a non transient production protocol. Thus, high vector titres can be produced from stable packaging cell lines which retain the vectors' self-inactivating properties in target cells that do not express tTA.

Thus, in one embodiment of the present invention, preferably the SIN vector is a conditional SIN (cSIN) vector.

Pseudotyping

In the design of viral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

As used herein, the term “pseudotyping” refers to a a technique or strategy whereby an env gene is replaced with a heterologous env gene. Pseudotyping is not a new phenomenon and examples may be found in WO-A-98/05759, WO-A-98/05754, WO-A-97/17457, WO-A-96/09400, WO-A-91/00047 and Mebatsion et al 1997 Cell 90, 841-847. Thus, the term “pseudotype” refers to progeny virions bearing the genome of one virus encapsidated by the envelope protein of another.

Pseudotyping can improve lentiviral vector stability and transduction efficiency. A pseudotype of murine leukemia virus packaged with lymphocytic choriomeningitis virus (LCMV) has been described (Miletic et al (1999) J. Virol. 73:6114-6116) and shown to be stable during ultracentrifugation and capable of infecting several cell lines from different species.

Preferably the env protein is an LCMV env protein.

In one embodiment of the present invention the vector system may be pseudotyped with at least a part of a rabies G protein or a mutant, variant, homologue or fragment thereof, or at least a part of a VSV G protein or a mutant, variant, homologue or fragment thereof.

The heterologous env region may be encoded by a gene which is present on a producer plasmid. The producer plasmid may be present as part of a kit for the production of lentiviral vector particles.

Rabies G Protein

In one embodiment of the present invention the vector system may be pseudotyped with at least a part of a rabies G protein or a mutant, variant, homologue or fragment thereof.

Teachings on the rabies G protein, as well as mutants thereof, may be found in in WO 99/61639 and well as Rose et al., 1982 J. Virol. 43: 361-364, Hanham et al., 1993 J. Virol.,67, 530-542, Tuffereau et al.,1998 J. Virol., 72, 1085-1091, Kucera et al., 1985 J. Virol 55, 158-162, Dietzschold et al., 1983 PNAS 80, 70-74, Seif et al., 1985 J.Virol., 53, 926-934, Coulon et al.,1998 J. Virol., 72, 273-278, Tuffereau et al.,1998 J. Virol., 72, 1085-10910, Burger et al., 1991 J.Gen. Virol. 72.359-367, Gaudin et al 1995 J Virol 69, 5528-5534, Benmansour et al 1991 J Virol 65, 4198-4203, Luo et al 1998 Microbiol Immunol 42, 187-193, Coll 1997 Arch Virol 142, 2089-2097, Luo et al 1997 Virus Res 51, 35-41, Luo et al 1998 Microbiol Immunol 42, 187-193, Coll 1995 Arch Virol 140, 827-851, Tuchiya et al 1992 Virus Res 25, 1-13, Morimoto et al 1992 Virology 189, 203-216, Gaudin et al 1992 Virology 187, 627-632, Whitt et al 1991 Virology 185, 681-688, Dietzschold et al 1978 J Gen Virol 40, 131-139, Dietzschold et al 1978 Dev Biol Stand 40, 45-55, Dietzschold et al 1977 J Virol 23, 286-293, and Otvos et al 1994 Biochim Biophys Acta 1224, 68-76. A rabies G protein is also described in EP-A-0445625.

The use of rabies G protein provides vectors which, in vivo, preferentially transduce targeted cells which rabies virus preferentially infects. This includes in particular neuronal target cells in vivo. For a neuron-targeted vector, rabies G from a pathogenic strain of rabies such as ERA may be particularly effective. On the other hand rabies G protein confers a wider target cell range in vitro including nearly all mammalian and avian cell types tested (Seganti et al., 1990 Arch Virol. 34,155-163; Fields et al., 1996 Fields Virology, Third Edition, vol.2, Lippincott-Raven Publishers, Philadelphia, N.Y.) .

The tropism of the pseudotyped vector particles may be modified by the use of a mutant rabies G which is modified in the extracellular domain. Rabies G protein has the advantage of being mutatable to restrict target cell range. The uptake of rabies virus by target cells in vivo is thought to be mediated by the acetylcholine receptor (AchR) but there may be other receptors to which in binds in vivo (Hanham et al., 1993 J. Virol.,67, 530-542; Tuffereau et al.,1998 J. Virol., 72, 1085-1091). It is thought that multiple receptors are used in the nervous system for viral entry, including NCAM (Thoulouze et al (1998) J. Virol 72(9):7181-90) and p75 Neurotrophin receptor (Tuffereau C et al (1998) Embo J 17(24) 7250-9).

The effects of mutations in antigenic site III of the rabies G protein on virus tropism have been investigated, this region is not thought to be involved in the binding of the virus to the acetylcholine receptor (Kucera et al., 1985 J. Virol 55, 158-162; Dietzschold et al., 1983 Proc Natl Acad Sci 80, 70-74; Seif et al., 1985 J.Virol., 53, 926-934; Coulon et al., 998 J. Virol., 72, 273-278; Tuffereau et al.,1998 J. Virol., 72, 1085-10910). For example a mutation of the arginine at amino acid 333 in the mature protein to glutamine can be used to restrict viral entry to olfactory and peripheral neurons in vivo while reducing propagation to the central nervous system. These viruses were able to penetrate motor neurons and sensory neurons as efficiently as the wild type virus, yet transneuronal transfer did not occur (Coulon et al., 1989, J. Virol. 63, 3550-3554). Viruses in which amino acid 330 has been mutated are further attenuated, being unable to infect either motor neurons or sensory neurons after intra-muscular injection (Coulon et al.,1998 J. Virol., 72, 273-278).

Alternatively or additionally, rabies G proteins from laboratory passaged strains of rabies may be used. These can be screened for alterations in tropism. Such strains include the following:

| Genbank accession number | Rabies Strain |
|---|---|
| J02293 | ERA |
| U52947 | COSRV |
| U27214 | NY 516 |
| U27215 | NY771 |
| U27216 | FLA125 |
| U52946 | SHBRV |
| M32751 | HEP-Flury |

By way of example, the ERA strain is a pathogenic strain of rabies and the rabies G protein from this strain can be used for transduction of neuronal cells. The sequence of rabies G from the ERA strains is in the GenBank database (accession number J02293). This protein has a signal peptide of 19 amino acids and the mature protein begins at the lysine residue 20 amino acids from the translation initiation methionine. The HEP-Flury strain contains the mutation from arginine to glutamine at amino acid position 333 in the mature protein which correlates with reduced pathogenicity and which can be used to restrict the tropism of the viral envelope.

WO 99/61639 discloses the nucleic and amino acid sequences for a rabies virus strain ERA (Genbank locus RAVGPLS, accession M38452).

VSV-G Protein

The envelope glycoprotein (G) of Vesicular stomatitis virus (VSV), a rhabdovirus, is another envelope protein that has been shown to be capable of pseudotyping certain lentiviruses.

Its ability to pseudotype MoMLV-based retroviral vectors in the absence of any retroviral envelope proteins was first shown by Emi et al (1991 Journal of Virology 65:1202-1207). WO94/294440 teaches that retroviral vectors may be successfully pseudotyped with VSV-G. These pseudotyped VSV-G vectors may be used to transduce a wide range of mammalian cells. Even more recently, Abe et al (J Virol 1998 72(8) 6356-6361) teach that non-infectious retroviral particles can be made infectious by the addition of VSV-G.

Burns et al (1993 Proc. Natl. Acad. Sci. USA 90: 8033-7) successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al 1993 ibid). They have also been shown to be more efficient than traditional amphotropic envelopes for a variety of cell lines (Yee et al, 1994 Proc. Natl. Acad. Sci. USA 91: 9564-9568, Lin, Emi et al, 1991 Journal of Virology 65:1202-1207). VSV-G protein can be used to pseudotype certain lentiviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al, 1996 J. Virol. 70: 2581-5). Lentivirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages.

WO00/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

Mutants, Variants, Homologues and Fragments

In one embodiment, the lentiviral vector system used in the present invention may be pseudotyped with a mutant, variant, homologue or fragment of the wild-type Rabies G or VSV-G protein.

The term “wild type” is used to mean an polypeptide having a primary amino acid sequence which is identical with the native protein (i.e., the viral protein).

The term “mutant” is used to mean a polypeptide having a primary amino acid sequence which differs from the wild type sequence by one or more amino acid additions, substitutions or deletions. A mutant may arise naturally, or may be created artificially (for example by site-directed mutagenesis).Preferably the mutant has at least 90% sequence identity with the wild type sequence. Preferably the mutant has 20 mutations or less over the whole wild-type sequence. More preferably the mutant has 10 mutations or less, most preferably 5 mutations or less over the whole wild-type sequence.

The term “variant” is used to mean a naturally occurring polypeptide which differs from a wild-type sequence. A variant may be found within the same viral strain (i.e. if there is more than one isoform of the protein) or may be found within a different strains. Preferably the variant has at least 90% sequence identity with the wild type sequence. Preferably the variant has 20 mutations or less over the whole wild-type sequence. More preferably the variant has 10 mutations or less, most preferably 5 mutations or less over the whole wild-type sequence.

Here, the term “homologue” means an entity having a certain homology with the wild type amino acid sequence and the wild type nucleotide sequence. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | GAP |
| | | ILV |
| | Polar - uncharged | CSTM |
| | | NQ |
| | Polar - charged | DE |
| | | KR |
| AROMATIC | | HFWY |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alalnine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*. p-nitro-L-phenylalanine*, L-hydroxyproline^{#}, L-thioproline*, methyl derivatives of phenylalanine (Phe) such as 4-methyl-Phe* pentamethyl-Phe*, L-Phe (4-amino)^{#}, L-Tyr (methyl)*, L-Phe (4-isopropyl)*, L-Tie (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term “fragment” indicates that the polypeptide comprises a fraction of the wild-type amino acid sequence. It may comprise one or more large contiguous sections of sequence or a plurality of small sections. The polypeptide may also comprise other elements of sequence, for example, it may be a fusion protein with another protein. Preferably the polypeptide comprises at least 50%, more preferably at least 65%, most preferably at least 80% of the wild-type sequence.

With respect to function, the mutant, variant, homologue or fragment should be capable of transducing the target cell when used to pseudotype an appropriate vector.

The viral delivery system used in the present invention may comprise nucleotide sequences that can hybridise to the nucleotide sequence presented herein (including complementary sequences of those presented herein). In a preferred aspect, the present invention covers nucleotide sequences that can hybridise to the nucleotide sequence of the present invention under stringent conditions (e.g. 65° C. and 0.1 SSC) to the nucleotide sequence presented herein (including complementary sequences of those presented herein).

A potential advantage of using the rabies glycoprotein in comparison to the VSV glycoprotein is the detailed knowledge of its toxicity to man and other animals due to the extensive use of rabies vaccines. In particular phase 1 clinical trials have been reported on the use of rabies glycoprotein expressed from a canarypox recombinant virus as a human vaccine (Fries et al., 1996 Vaccine 14, 428-434), these studies concluded that the vaccine was safe for use in humans.

Hybrid Viral Vectors

In a further broad aspect, the present invention provides a hybrid viral vector system for in vivo delivery of a nucleotide sequence encoding an NOI of the present invention, which system comprises one or more primary viral vectors which encode a secondary viral vector, the primary vector or vectors capable of infecting a first target human cell and of expressing therein the secondary viral vector, which secondary vector is capable of transducing a secondary target cell.

Preferably the primary vector is obtainable from or is based on an alphaviral vector and/or the secondary viral vector is obtainable from or is based on a lentiviral vector.

Regulated Viral Vectors

In one aspect of the present invention, the alpha-lentiviral vector, lentiviral component (referred to in this discussion also as lentiviral vector for ease only) or lentiviral vector is a regulated.

As used herein, the term “regulated viral vectors” means a viral vector comprising a “regulatable 3′LTR region. As used herein, the terms “regulatable LTR” and “regulatable 3′LTR” include vectors which contain responsive elements which are present in lentiviral 3′ LTR sequences, either by design or by their nature. Within the regulatable 3′LTR region, the 3′U3 sequence contains most of the transcriptional control elements of the provirus, which include the promoter and multiple enhancer sequences responsive to cellular and in some cases, viral transcriptional activator proteins.

Viral Delivery Systems

When the lentivector particles are used to transfer NOIs into cells which they transduce, such lentivector particles also designated “viral delivery systems” or “lentiviral delivery systems”. Viral vectors, including lentiviral vectors, have been used to transfer NOIs efficiently by exploiting the viral transduction process. NOIs cloned into the lentiviral genome can be delivered efficiently to cells susceptible to transduction by a lentivirus. Through other genetic manipulations, the replicative capacity of the lentiviral genome can be destroyed. The vectors introduce new genetic material into a cell but are unable to replicate.

Preferably the genome of the vector system used in the present invention comprises a cPPT sequence.

The presence of a sequence termed the central polypurine tract (cPPT) may improve the efficiency of gene delivery to non-dividing cells (see WO 00/31200). This cis-acting element is located, for example, in the EIAV polymerase coding region element.

High Titre

It is highly desirable to use high-titre virus preparations in both experimental and practical applications. Techniques for increasing viral titre include using a psi plus packaging signal, as discussed above, and concentration of viral stocks.

As used herein, the term “high titre” means an effective amount of a viral vector or particle which is capable of transducing a target site such as a cell.

As used herein, the term “effective amount” means an amount of a regulated viral or vector particle which is sufficient to induce expression of the NOIs at a target site. In some instances, the term “sufficient amount” is used interchangeably with the term “effective amount”.

A high-titre viral preparation for a producer/packaging cell is usually of the order of 10⁵ to 10⁷ t.u. per ml. (The titer is expressed in transducing units per ml (t.u./ml) as titred on the canine osteosarcoma D17 cell line). For transduction in tissues such as the brain, it is necessary to use very small volumes, so the viral preparation is concentrated by ultracentrifugation. The resulting preparation should have at least 10⁸ t.u./ml, preferably from 10⁸ to 10⁹ t.u./ml, more preferably at least 10⁹ t.u./ml.

Preferably the lentiviral vector is produced at high titre.

Preferably a recombinase assisted mechanism is used which facilitates the production of high titre regulated lentiviral vectors from the producer cells of the present invention.

As used herein, the term “recombinase assisted system” includes but is not limited to a system using the Cre recombinase/1oxP recognition sites of bacteriophage P1 or the site-specific FLP recombinase of S. cerevisiae which catalyses recombination events between 34 bp FLP recognition targets (FRTs).

The site-specific FLP recombinase of S. cerevisiae which catalyses recombination events between 34 bp FLP recognition targets (FRTs) has been configured into DNA constructs in order to generate high level producer cell lines using recombinase-assisted recombination events (Karreman et al (1996) NAR 24:1616-1624). A similar system has been developed using the Cre recombinase/loxP recognition sites of bacteriophage P1 (see PCT/GB00/03837; Vanin et al (1997) J. Virol 71:7820-7826). This was configured into a lentiviral genome such that high titre lentiviral producer cell lines were generated.

Preferably a high titre lentiviral vector is produced using a modified and/or extended packaging signal.

Packaging Signal

As used herein, the term “packaging signal” which is refered to interchangeably as “packaging sequence” or “psi” is used in reference to the non-coding sequence located within the lentiviral genome which is required for encapsidation of lentiviral RNA strands during viral particle formation. In HIV-1, this sequence has been mapped to loci extending from upstream of the major splice donor site (SD) to at least the gag start codon. Several lentiviral vectors use the minimal packaging signal (also referred to as the psi sequence) needed for encapsidation of the viral genome. By way of example, this minimal packaging signal encompasses bases 212 to 563 of the Mo-MLV genome (Mann et al 1983: Cell 33: 153

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Preferably a high titre lentiviral vector is produced using a modified packaging signal.

Preferably the lentiviral construct is a based on an EIAV vector genome where all the accessory genes are removed.

Accessory Genes

As used herein, the term “accessory genes” refer to a variety of virally encoded accessory proteins capable of modulating various aspects of lentiviral replication and infectivity. These proteins are discussed in Coffin et al (ibid) (Chapters 6 and 7). Examples of accessory proteins in lentiviral vectors include but are not limited to tat, rev, nef, vpr, vpu, vif, vpx. An example of a lentiviral vector useful in the present invention is one which has all of the accessory genes removed.

Transcriptional Control

The control of proviral transcription remains largely with the noncoding sequences of the viral LTR. The site of transcription initiation is at the boundary between U3 and R in the 5′LTR (as shown in FIG. 31) and the site of poly (A) addition (termination) is at the boundary between R and U5 in the 3′LTR (as shown in FIG. 31). The U3 sequence contains most of the transcriptional control elements of the provirus, which include the promoter and multiple enhancer sequences responsive to cellular and in some cases, viral transcriptional activator proteins.

An LTR present, for example, in a construct of the present invention and as a 3′LTR in the provirus of, for example, a target cell of the invention may be a native LTR or a heterologous regulatable LTR. It may also be a transcriptionally quiescent LTR for use in SIN vector technology.

The term “regulated LTR” also includes an inactive LTR such that the resulting provirus in the target cell can not produce a packagable viral genome (self-inactivating (SIN) vector technology).

Preferably the regulated lentiviral vector of the present invention is a self-inactivating (SIN) vector.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Preferably the targeted vector comprises a nucleotide sequence of interest (NOI) for delivery to a specific cell type.

Nucleotide Sequence of Interest (NOI)

With the present invention, the term NOI (i.e. nucleotide sequence of interest) includes any suitable nucleotide sequence, which need not necessarily be a complete naturally occuring DNA sequence. Thus, the DNA sequence can be, for example, a synthetic DNA sequence, a recombinant DNA sequence (i.e. prepared by use of recombinant DNA techniques), a cDNA sequence or a partial genomic DNA sequence, including combinations thereof. The DNA sequence need not be a coding region. If it is a coding region, it need not be an entire coding region. In addition, the DNA sequence can be in a sense orientation or in an anti-sense orientation. Preferably, it is in a sense orientation. Preferably, the DNA is or comprises cDNA.

The NOI or NOIs may be under the expression control of an expression regulatory element, usually a promoter or a promoter and enhancer. The enhancer and/or promoter may be preferentially active in a hypoxic or ischaemic or low glucose environment, such that the NOI is preferentially expressed in the particular tissues of interest, such as in the environment of a tumour, arthritic joint or other sites of ischaemia. Thus any significant biological effect or deleterious effect of the NOI on the individual being treated may be reduced or eliminated. The enhancer element or other elements conferring regulated expression may be present in multiple copies. Likewise, or in addition, the enhancer and/or promoter may be preferentially active in one or more specific cell types—such as any one or more of macrophages, endothelial cells or combinations thereof. Further examples include include respiratory airway epithelial cells, hepatocytes, muscle cells, cardiac myocytes, synoviocytes, primary mammary epithelial cess and post-mitotically terminally differentiated non-replicating cells such as macrophages neurons.

NOIs

In accordance with the present invention, suitable NOI sequences include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives therof (such as with an associated reporter group). When included, such coding sequences may be typically operatively linked to a suitable promoter, which may be a promoter driving expression of a ribozyme(s), or a different promoter or promoters, such as in one or more specific cell types.

Suitable NOIs for use in the invention in the treatment or prophylaxis of cancer include NOIs encoding proteins which: destroy the target cell (for example a ribosomal toxin), act as: tumour suppressors (such as wild-type p53); activators of anti-tumour immune mechanisms (such as cytokines, co-stimulatory molecules and immunoglobulins); inhibitors of angiogenesis; or which provide enhanced drug sensitivity (such as pro-drug activation enzymes); indirectly stimulate destruction of target cell by natural effector cells (for example, strong antigen to stimulate the immune system or convert a precursor substance to a toxic substance which destroys the target cell (for example a prodrug activating enzyme). Encoded proteins could also destroy bystander tumour cells (for example with secreted antitumour antibody-ribosomal toxin fusion protein), indirectly stimulated destruction of bystander tumour cells (for example cytokines to stimulate the immune system or procoagulant proteins causing local vascular occlusion) or convert a precursor substance to a toxic substance which destroys bystander tumour cells (eg an enzyme which activates a prodrug to a diffusible drug).

Also, the delivery of NOI(s) encoding antisense transcripts or ribozymes which interfere with expression of cellular genes for tumour persistence (for example against aberrant myc transcripts in Burkitts lymphoma or against bcr-abl transcripts in chronic myeloid leukemia. The use of combinations of such NOIs is also envisaged.

Suitable NOIs for use in the treatment or prevention of ischaemic heart disease include NOIs encoding plasminogen activators. Suitable NOIs for the treatment or prevention of rheumatoid arthritis or cerebral malaria include genes encoding anti-inflammatory proteins, antibodies directed against tumour necrosis factor (TNF) alpha, and anti-adhesion molecules (such as antibody molecules or receptors specific for adhesion molecules).

Examples of hypoxia regulatable therapeutic NOIs can be found in PCT/GB95/00322 (WO-A-9521927).

The expression products encoded by the NOIs may be proteins which are secreted from the cell. Alternatively the NOI expression products are not secreted and are active within the cell. In either event, it is preferred for the NOI expression product to demonstrate a bystander effector or a distant bystander effect; that is the production of the expression product in one cell leading to the killing of additional, related cells, either neighbouring or distant (e.g. metastatic), which possess a common phenotype.

The NOI or NOIs of the present invention may also comprise one or more cytokine-encoding NOIs. Suitable cytokines and growth factors include but are not limited to: ApoE, Apo-SAA, BDNF, Cardiotrophin-1, EGF, ENA-78, Eotaxin, Eotaxin-2, Exodus-2, FGF-acidic, FGF-basic, fibroblast growth factor-10 (Marshall 1998 Nature Biotechnology 16: 129).FLT3 ligand (Kimura et al (1997), Fractalkine (CX3C), GDNF, G-CSF, GM-CSF, GF-βT, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein (Marshall 1998 ibid), M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β, GRO-γ, HCCl, 1-309.

Target Cells

Target cells transduced by the alpha-lentiviral vector of the present invention may be used to express the NS/lentiviral vector of the present invention under in vitro, in vivo and ex vivo conditions. In addition, target cells transduced by the NS/lentiviral vector of the present invention may be used to express the NOI of the present invention under in vitro, in vivo and ex vivo conditions. The former target cell is commonly referred to as the “primary” or “host” target cell, and the latter as the “secondary” target, or simply “target” cell.

The term “target cell” includes any cell derivable from a suitable organism which a vector is capable of transfecting or transducing. Examples of target cells can include but are not limited to cells capable of expressing the lentiviral vector and/or NOI of the present invention under in vitro, in vivo and ex vivo conditions. Examples of such cells include but are not limited to macrophages, endothelial cells or combinations thereof. Further examples include but are not limited to hematopoietic stem cells, lymphocytes, vascular endothelial cells, respiratory epithelial cells, keratinocytes, skeletal and cardiac muscle cells, neurons, cancer cells respiratory airway epithelial cells, hepatocytes, muscle cells, cardiac myocytes, synoviocytes, primary mammary epithelial cells and post-mitotically terminally differentiated non-replicating cells such as macrophages and/or neurons.

In the present invention the target cell of the alpha-lentiviral vector is a human cell.

In a preferred embodiment, the target cell of the lentiviral vector is a mammalian cell.

In a highly preferred embodiment, the target cell of the lentiviral vector is a human cell.

The term “organism” includes any suitable organism. In a preferred embodiment, the organism is a mammal. In a highly preferred embodiment, the organism is a human.

The present invention also provides a method comprising transforming a host cell (such as a human packaging/producer cell) with a NS(s) of the present invention and/or target cell with a lentiviral vector comprising a NOI of the present invention

The term “transformed cell” means a host cell and/or a target cell having a modified genetic structure. With the present invention, a cell has a modified genetic structure when a vector comprising an NOI according to the present invention has been introduced into the cell.

Host cells and/or a target cells may be cultured under suitable conditions which allow expression of the NS and/or NOI of the present invention.

The present invention also provides a method comprising culturing a transformed host cell—which cell has been transformed with one or more NS (s) according to the present invention and/or under conditions suitable for the expression of a POI encoded by an NOI.

Regulation of Expression in vitro/vivo/ex vivo

The present invention also encompasses gene delivery using a lentiviral vector whereby the expression of the NOI is regulated in vitrolin vivolex vivo. For example, expression regulation may be accomplished by administering compounds that bind to the NOI, or control regions associated with the NOI or its corresponding RNA transcript to modify the rate of transcription or translation. The present invention also encompasses production of lentiviral vectors using alpha-lentiviral vectors whereby expression of the lentiviral vector is regulated in vitrolin vivolex vivo.

Control Sequences

Control sequences operably linked to sequences encoding the NS or NOI include promoters/enhancers and other expression regulation signals. These control sequences may be selected to be compatible with the host cell and/or target cell in which an expression vector comprising an NS and/or a viral vector comprising an NOI is designed to be used. The control sequences may be modified, for example by the addition of further transcriptional regulatory elements to make the level of transcription directed by the control sequences more responsive to transcriptional modulators.

Operably Linked

The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Preferably the nucleotide sequence of the present invention is operably linked to a transcription unit.

The term “transcription unit(s)” as described herein are regions of nucleic acid containing coding sequences and the signals for achieving expression of those coding sequences independently of any other coding sequences. Thus, each transcription unit generally comprises at least a promoter, an optional enhancer and a polyadenylation signal.

Promoters

The term promoter is well-known in the art and is used in the normal sense of the art, e.g. an RNA polymerase binding site. The term encompasses nucleic acid regions ranging in size and complexity from minimal promoters to promoters including upstream elements and enhancers.

The promoter is typically selected from promoters which are functional in mammalian, cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase).

Preferably the promoter is a constitutive promoter such as CMV.

Preferably the promoters of the present invention are tissue specific.

Hypoxic Promoters/Enhancers

The enhancer and/or promoter may be preferentially active in a hypoxic or ischaemic or low glucose environment, such that an NS or NOI is preferentially expressed in the particular tissues of interest, such as in the environment of a tumour, arthritic joint or other sites of ischaemia. Thus, any significant biological effect or deleterious effect of the expressed NS or NOI on the individual being treated may be reduced or eliminated. The enhancer element or other elements conferring regulated expression may be present in multiple copies. Likewise, or in addition, the enhancer and/or promoter may be preferentially active in one or more specific cell types—such as any one or more of macrophages, endothelial cells or combinations thereof. Further examples may include but are not limited to respiratory airway epithelial cells, hepatocytes, muscle cells, cardiac myocytes, synoviocytes, primary mammary epithelial cells and post-mitotically terminally differentiated non-replicating cells such as macrophages and/or neurons.

Tissue-Specific Promoters

The promoters of the present invention may be tissue-specific promoters. Examples of suitable tissue restricted promoters/enhancers are those which are highly active in tumour cells such as a promoter/enhancer from a MUCI gene, a CEA gene or a 5T4 antigen gene. Examples of temporally restricted promoters/enhancers are those which are responsive to ischaemia and/or hypoxia, such as hypoxia response elements or the promoter/enhancer of a grp78 or a grp94 gene. The alpha fetoprotein (AFP) promoter is also a tumour-specific promoter. One preferred promoter-enhancer combination is a human cytomegalovirus (hCMV) major immediate early (MIE) promoter/enhancer combination.

Preferably the promoters of the present invention are tissue specific. That is, they are capable of driving transcription of an NS or NOI (s) in one tissue while remaining largely “silent” in other tissue types.

The term “tissue specific” means a promoter which is not restricted in activity to a single tissue type but which nevertheless shows selectivity in that they may be active in one group of tissues and less active or silent in another group. A desirable characteristic of the promoters of the present invention is that they possess a relatively low activity in the absence of activated hypoxia-regulated enhancer elements, even in the target tissue. One means of achieving this is to use “silencer” elements which suppress the activity of a selected promoter in the absence of hypoxia.

The level of expression of an NS or NOI under the control of a particular promoter may be modulated by manipulating the promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity or the smallest region conferring hypoxia sensitivity.

A number of tissue specific promoters, described above, may be particularly advantageous in practising the present invention. In most instances, these promoters may be isolated as convenient restriction digestion fragments suitable for cloning in a selected vector. Alternatively, promoter fragments may be isolated using the polymerase chain reaction. Cloning of the amplified fragments may be facilitated by incorporating restriction sites at the 5′ end of the primers.

Preferably the ischaemic responsive promoter is a tissue restricted ischaemic responsive promoter.

Preferably the tissue restricted ischaemic responsive promoter is a macrophage specific promoter restricted by repression.

Preferably the tissue restricted ischaemic responsive promoter is an endothelium specific promoter.

Preferably the tissue restricted ischaemic responsive promoter of the present invention is an ILRE responsive promoter.

Preferably the vector comprising ILRE responsive promoter is a lentiviral vector.

Preferably the vector comprising ILRE responsive promoter is an autoregulated hypoxia responsive lentiviral vector.

Preferably the vector of the present invention is regulated by glucose concentration.

For example, the glucose-regulated proteins (grp's) such as grp78 and grp94 are highly conserved proteins known to be induced by glucose deprivation (Attenello and Lee 1984 Science 226 187-190). The grp 78 gene is expressed at low levels in most normal healthy tissues under the influence of basal level promoter elements but has at least two critical “stress inducible regulatory elements” upstream of the TATA element (Attenello 1984 ibid; Gazit et al 1995 Cancer Res 55: 1660-1663). Attachment to a truncated 632 base pair sequence of the 5′end of the grp78 promoter confers high inducibility to glucose deprivation on reporter genes in vitro (Gazit et al 1995 ibid). Furthermore, this promoter sequence in lentiviral vectors was capable of driving a high level expression of a reporter gene in tumour cells in murine fibrosarcomas, particularly in central relatively ischaemic/fibrotic sites (Gazit et al 1995 ibid).

Inducible Promoters

The promoters of the present invention may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Enhancer

In addition, any of these promoters may be modified by the addition of further regulatory sequences, for example enhancer sequences. Chimeric promoters may also be used comprising sequence elements from two or more different promoters described above.

The term “enhancer” includes a DNA sequence which binds to other protein components of the transcription initiation complex and thus facilitates the initiation of transcription directed by its associated promoter.

The in vitro/in vivo/ex vivo expression of an NOI may be used in combination with a protein of interest (POI) or a nucleotide sequence of interest (NOI) encoding same.

ILRE

The term “ischaemia like response element”—otherwise written as ILRE—includes an element that is responsive to or is active under conditions of ischaemia or conditions that are like ischaemia or are caused by ischaemia. By way of example, conditions that are like ischaemia or are caused by ischaemia include hypoxia and/or low glucose concentration(s).

Ischaemia can be an insufficient supply of blood to a specific organ or tissue. A consequence of decreased blood supply is an inadequate supply of oxygen to the organ or tissue (hypoxia). Prolonged hypoxia may result in injury to the affected organ or tissue.

A preferred ILRE is an hypoxia response element (HRE).

HRE

In one preferred aspect of the present invention, there is hypoxia or ischaemia regulatable expression of the lentiviral vector components. In this regard, hypoxia is a powerful regulator of gene expression in a wide range of different cell types and acts by the induction of the activity of hypoxia-inducible transcription factors such as hypoxia inducible factor-1 (HIF-1; Wang & Semenza 1993 Proc Natl Acad Sci 90:430), which bind to cognate DNA recognition sites, the hypoxia-responsive elements (HREs) on various gene promoters. Dachs et al (1997 Nature Med 5: 515) have used a multimeric form of the HRE from the mouse phosphoglycerate kinase-1 (PGK-1) gene (Firth et al 1994 Proc Natl Acad Sci 91:6496-6500) to control expression of both marker and therapeutic genes by human fibrosarcoma cells in response to hypoxia in vitro and within solid tumours in vivo (Dachs et al ibid).

Hypoxia response enhancer elements (HREEs) have also been found in association with a number of genes including the erythropoietin (EPO) gene (Madan et al 1993 Proc Natl Acad Sci 90: 3928; Semenza and Wang 1992 Mol Cell Biol 1992 12: 5447-5454). Other HREEs have been isolated from regulatory regions of both the muscle glycolytic enzyme pyrivate kinase (PKM) gene (Takenaka et al 1989 J Biol Chem 264: 2363-2367), the human muscle-specific β-enolase gene (ENO3; Peshavaria and Day 1991 Biochem J 275: 427-433) and the endothelin-1 (ET-1) gene (Inoue et al 1989 J Biol Chem 264: 14954-14959).

Preferably the HRE of the present invention is selected from, for example, the erythropoietin HRE element (HREE1), muscle pyruvate kinase (PKM), HRE element, phosphoglycerate kinase (PGK) HRE, β-enolase (enolase 3; ENO3) HRE element, endothelin-1 (ET-1)HRE element and metallothionein II (MTII) HRE element.

Responsive Element

Preferably the ILRE is used in combination with a transcriptional regulatory element, such as a promoter, which transcriptional regulatory element is preferably active in one or more selected cell type(s), preferably being only active in one cell type.

This combination aspect of the present invention is called a responsive element.

Preferably the responsive element comprises at least the ILRE as herein defined.

Non-limiting examples of such a responsive element are presented as OBHRE1 and XiaMac. Another non-limiting example includes the ILRE in use in conjunction with an MLV promoter and/or a tissue restricted ischaemic responsive promoter. These responsive elements are disclosed in WO99/15684.

Other examples of suitable tissue restricted promoters/enhancers are those which are highly active in tumour cells such as a promoter/enhancer from a MUC1 gene, a CEA gene or a 5T4 antigen gene. The alpha fetoprotein (AFP) promoter is also a tumour-specific promoter. One preferred promoter-enhancer combination is a human cytomegalovirus (hCMV) major immediate early (MIE) promoter/enhancer combination.

In one embodiment of the present invention, preferably the responsive elemtn is an ecdysone response element (see WO 97/38117 and WO 99/58155).

Combination with POIs/NOIs

The POI or NOI encoding same may be used in combination with a POI, such as a pro-drug activating enzyme either directly or by vector delivery to, for example, a target cell or target such as an ischaemic target tissue. Instead of or as well as being selectively expressed in target tissues, the POI or NOI encoding same may be used in combination with another POI such as a pro-drug activation enzyme or enzymes or with a nucleotide sequences of interest (NOI) or NOIs which encode a pro-drug activation enzyme or enzymes. These pro-drug activation enzyme or enzymes may have no significant effect or no deleterious effect until the individual is treated with one or more pro-drugs upon which the appropriate pro-drug enzyme or enzymes act. In the presence of the active POI or NOI encoding same, treatment of an individual with the appropriate pro-drug may lead to enhanced reduction in the disease condition such as a reduction in tumour growth or survival.

Pro-Drug POIS

A POI, such as a pro-drug activating enzyme, may be delivered to a disease site, such as a tumour site for the treatment of a cancer. In each case, a suitable pro-drug is used in the treatment of the patient in combination with the appropriate pro-drug activating enzyme. An appropriate pro-drug may be administered in conjunction with the enzyme or vector comprising the nucleotide sequence encoding same. Examples of pro-drugs include: etoposide phosphate (with alkaline phosphatase, Senter et a/1988 Proc Natl Acad Sci 85: 4842-4846); 5-fluorocytosine (with cytosine deaminase, Mullen et al1994 Cancer Res 54: 1503-1506); Doxorubicin-N-p-hydroxyphenoxyacetamide (with Penicillin-V-Amidase, Kerr et al1990 Cancer Immunol Immunother 31: 202-206); Para-N-bis(2-chloroethyl) aminobenzoyl glutamate (with carboxypeptidase G2); Cephalosporin nitrogen mustard carbamates (with Pb-lactamase); SR4233 (with P450 Reductase); Ganciclovir (with HSV thymidine kinase, Borrelli et al 1988 Proc Natl Acad Sci 85: 7572-7576); mustard pro-drugs with nitroreductase (Friedlos et al1997 J Med Chem 40: 1270-1275) and Cyclophosphamide (with P450 Chen et al1996 Cancer Res 56: 1331-1340).

Examples of suitable pro-drug activation enzymes for use in the invention include a thymidine phosphorylase which activates the 5-fluoro-uracil pro-drugs capcetabine and furtulon; thymidine kinase from Herpes Simplex Virus which activates ganciclovir; a cytochrome P450 which activates a pro-drug such as cyclophosphamide to a DNA damaging agent; and cytosine deaminase which activates 5-fluorocytosine. Preferably, a pro-drug activating enzyme of human origin is used. POIs AND NOIs

Other suitable proteins of interest (POIs) or NOIs encoding same for use in the present invention include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives therof (such as with an associated reporter group). When included, the POIs or NOIs encoding same may be typically operatively linked to a suitable promoter, which may be a promoter driving expression of a ribozyme(s), or a different promoter or promoters, such as in one or more specific cell types.

Cytokines

In one aspect of the present invention the NOI(s) encodes a POI(s) wherein the POI is a cytokine.

As used herein, the term “cytokines” refers to any varied group of proteins that are released from mammalian cells and act on other cells through specific receptors. The term “cytokine” is often used interchangeably with the term “mediator”. Cytokines may elicit from the target cell a variety of responses depending on the cytokine and the target cell. By way of example, cytokines may be important in signalling between cells as inflammatory reactions develop. In the initial stages, cytokines such as IL-1 and IL-6 may be released from cells of the tissue where the inflammatory reaction is occurring. Once lymphocytes and mononuclear cells have started to enter the inflammatory site, they may become activated by antigen and release cytokines of their own such as IL-1, TNF, IL-4 and IFNγ which further enhance cellular migration by their actions on the local endothelium. Other cytokines, such as IL-8, are chemotactic or can activate incoming cells. The term “cytokine” includes but is not limited to factors such as cardiotrophin, EGF, FGF-acidic, FGF-basic, flt3 Ligand, G-CSF, GM-CSF, IFN-γ, IGF-I, IGF-II, IL-1α, IL-1β, IL-2, IL-3, IL-4, IL-5, IL-6, IL-7, IL-8, IL-9, IL-10, IL-11, IL-12, IL-13, IL-15, IL-16, IL-17, IL-18 (IGIF), KGF, LIF, M-CSF, Oncostatin M, PDGF-A, PDGF-AB, PDGF-BB, SCF, SCGF, TGF-α, TGF-β₁, TNF-α, TNF-β, TPO and VEGF.

Tumour Necrosis Factor (TNF)

Preferably the POI is TNF.

As used herein, the term “tumour necrosis factor (TNF) refers to either of two structurally and functionally related proteins. These are TNF-α (also known as cachectin) and TNF-β (also known as lymphotoxin) TNF-α is producted mainly by monocytes and macrophages, whereas TNF-β is produced by lymphoid cells. The two proteins are about 30% homologous at the amino-acid level, and bind to the same cell surface receptors; both exist as homotrimers. Both TNF-α (cachectin) and TNF-β (lymphotoxin) were originally thought of as selective antitumour agents, but are now known to have a multiplicity of actions. In binding to their receptors, present on virtually all cells examined, they activate a large array of cellular genes and also multiple signal-transduction pathways, kinases, and transcription factors. Their genes are single-copy genes, closely linked within the MHC cluster.

Tumour Necrosis Factor α (TNF-α)

Preferably the POI is TNF-α.

As used herein, the term tumour necrosis factor α (TNF-α) (also known as cachectin) refers to a cytokine that is produced by macrophages, monocytes, endothelial cells, neutrophils, smooth muscle cells, activated lymphocytes, and astrocytes. It is a transmembrane glycoprotein and cytotoxin with a variety of functions, including the ability to mediate the expression of genes for growth factors, cytokines, transcription factors, and receptors. It can cause cytolysis of certain tumour cell lines, it has been implicated in the induction of cachexia (which is a condition caused by chronic disease, such as cancer), it is a potent pyrogen, causing fever by direct action or by stimulation of interleukin 1 secretion, and it can stimulate cell proliferation and induce cell differentiation under certain conditions. The molecule is a homotrimer. Inflammatory stimulators such as TNF-α also cause a rapid (<1 hour) inhibition of chemotactic migration of monocytes with similar kinetics to hypoxia. TNF-α increases HIF-1 binding to DNA and TNF-α is hypoxia-responsive in many cell types, including macrophages.

Chemokines

In one aspect of the present invention the POI is a chemokine As used herein, the term “chemokine” (also known as intercrine) refers to any of a superfamily of soluble proteins implicated in a wide range of acute and inflammatory processes and other immunoregulatory functions. The chemokines may related by primary structure, especially conservation of a motif of four cysteines, the first two of which are either adjacent of separated by one other residue. As used herein, the term “chemokines” includes a group of at least 18 heparin-binding molecules, including IL-8, which are released at inflammatory sites. These chemokines act via a group of three receptors (so far identified) that are expressed on different leucocyte populations (see Immunology 1996 4^{th} Ed Roitt Brostoff and Male, Mosby publishers, page Chapter 14, page 14.7). Some of the chemokines act selectively on particular populations of leucocytes. Some of the chemokines can activate cells, some are primarily chemotactic, some have both functions. Several inflammatory mediators may be chemotactic. By way of example, several molecules are chemotactic for neutrophils and macrophages. These molecules include C5a, f.Met-Leu-Phe, LTB4 which act on neutrophils, eosinophils and macrophages (see FIG. 14.12 Immunology 1996 4^{th} Ed Roitt Brostoff and Male, Mosby publishers, page Chapter 14, page 14.7). Other chemokines, such as IL-8, macrophage inflammatory protein alpha (MIP-α), inflammatory protein beta (MIP-β) and RANTES which have selective actions on different leucocyte populations. In this respect, the term “chemokines” includes but is not limited to factors such as ENA-78, Eotaxin, Eotaxin-2, Exodus-2, Fractalkine (CX3C), GCP-2, GRO/MGSA, GRO-β, GRO-γ, HCCl, 1-309, IL-8 (72 a.a.), IL-8 (77 a.a.), IP-10, Lymphotactin, MDC (67 a.a.), MDC (69a.a.), MCP-1 (MCAF), Human MCP-2, MCP-3, MCP-4, MDC (67 a.a.), MDC (69 a.a.), MIG, MIP-1α, MIP-1β, MIP-3α, MIP-3β, Human MIP-4, NAP-2, PF-4, RANTES, SDF1α, SDF1α, TARC, C-10, Eotaxin, Exodus-2, JE (MCP-1), KC, MCP-3, MCP-5, MIP-1α, MIP-1γ, RANTES, GROβ/MIP-2 and MCP-1(MCAF).

Bystander Effect

The POI and/or NOI encoding same may be proteins which are secreted from a cell. Alternatively the POI expression products are not secreted and are active within the cell. In either event, it is preferred for the POI expression product to demonstrate a bystander effector or a distant bystander effect; that is the production of the expression product in one cell leading to the killing of additional, related cells, either neighbouring or distant (e.g. metastatic), which possess a common phenotype.

Suitable POIs or NOIs encoding same for use in the present invention in the treatment or prophylaxis of cancer include proteins which: destroy the target cell (for example a ribosomal toxin), act as: tumour suppressors (such as wild-type p53); activators of anti-tumour immune mechanisms (such as cytokines, co-stimulatory molecules and immunoglobulins); inhibitors of angiogenesis; or which provide enhanced drug sensitivity (such as pro-drug activation enzymes); indirectly stimulate destruction of target cell by natural effector cells (for example, strong antigen to stimulate the immune system or convert a precursor substance to a toxic substance which destroys the target cell (for example a prodrug activating enzyme). Encoded proteins could also destroy bystander tumour cells (for example with secreted antitumour antibody-ribosomal toxin fusion protein), indirectly stimulate destruction of bystander tumour cells (for example cytokines to stimulate the immune system or procoagulant proteins causing local vascular occlusion) or convert a precursor substance to a toxic substance which destroys bystander tumour cells (eg an enzyme which activates a prodrug to a diffusible drug).

Also, the delivery of NOI(s) encoding antisense transcripts or ribozymes which interfere with expression of cellular genes for tumour persistence (for example against aberrant myc transcripts in Burkitts lymphoma or against bcr-abl transcripts in chronic myeloid leukemia. The use of combinations of such POIs and/or NOIs encoding same is also envisaged.

Examples of hypoxia regulatable therapeutic NOIs can be found in PCT/GB95/00322 (WO-A-95/2 1927).

Pharmaceutical Compositions

In one aspect, the present invention provides a pharmaceutical composition, which comprises a viral vector according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice.

The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

The invention further provides a method of preventing and/or treating a disorder, such as a cancer disorder in an individual, the method comprising, for example, administering to an individual an viral vector and/or pharmaceutical composition comprising same to a target site.

As used herein, the term “administered” includes but is not limited to delivery by a mucosal route, for example, as a nasal spray or aerosol for inhalation or as an ingestable solution such as by an oral route, or by a parenteral route where delivery is by an injectable form, such as, for example, by a rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, intracavemosal, subcutaneous, transdermal or intramuscular) route.

The viral vector and/or pharmaceutical composition comprising same of the present invention may be administered alone but will generally be administered in admixture with a suitable pharmaceutical excipient, diluent or carrier selected with regard to the intended route of administration and standard pharmaceutical practice.

For example, the viral vector and/or pharmaceutical composition or modified monocyte/macrophage comprising same can be administered orally, buccally or sublingually in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The viral vector and/or pharmaceutical composition comprising same can also be administered parenterally, for example, intravenously, intra-arterially, intraperitoneally, intrathecally, intraventricularly, intraurethrally, intrasternally, intracranially, intramuscularly or subcutaneously, or it may be administered by infusion techniques. For such parenteral administration it is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Thus tablets or capsules of the viral vector and/or pharmaceutical composition or comprising same may contain active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that, in the treatment of certain conditions the agent may be taken as a single dose as needed or desired.

The viral vector and/or pharmaceutical composition or modified monocyte/macrophage comprising same of the present invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the viral vector and/or pharmaceutical composition comprising same of the present invention can be administered in the form of a suppository or pessary, or it may be applied topically in the form of a gel, hydrogel, lotion, solution, cream, ointment or dusting powder. The viral vector and/or pharmaceutical composition comprising same of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Individual

As used herein, the term “individual” refers to vertebrates, particularly members of the mammalian species, more in particular, humans.

Treatment

It is to be appreciated that all references herein to treatment include curative, palliative and prophylactic treatment.

Combination Therapy

The viral vector and/or pharmaceutical compositio comprising same may be administered alone or in combination for the treatment of a disorder, such as a disorder associated with hypoxia and/or inflammation.

By way of further example, the viral vector and/or pharmaceutical composition may be administered with another agent, such as an NOI at the same moment in time and at the same site. Alternatively, viral vector and/or pharmaceutical composition comprising same may be delivered at a different time and to a different site. In one embodiment, the viral vector and/or pharmaceutical composition comprising same may even be delivered in the same delivery vehicle for the prevention and/or treatment of a disorder associated with hypoxia and/or inflammation.

Preferably the viral vector and/or pharmaceutical composition comprising same is/are administered simultaneously, separately or sequentially.

Dosage

The dosage of the viral vector and/or pharmaceutical composition comprising same of the present invention will depend on the disease state or condition being treated and other clinical factors such as weight and condition of the individual and the route of administration of the compound. Depending upon the half-life of the viral vector in the particular individual, the viral vector and/or pharmaceutical composition comprising same can be administered between several times per day to once a week. It is to be understood that the present invention has application for both human and veterinary use. The methods of the present invention contemplate single as well as multiple administrations, given either simultaneously or over an extended period of time.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

In addition or in the alternative the compositions (or component parts thereof) of the present invention may be administered by direct injection. In addition or in the alternative the compositions (or component parts thereof) of the present invention may be administered topically. In addition or in the alternative the compositions (or component parts thereof) of the present invention may be administered by inhalation. In addition or in the alternative the compositions (or component parts thereof) of the present invention may also be administered by one or more of: a mucosal route, for example, as a nasal spray or aerosol for inhalation or as an ingestable solution such as by an oral route, or by a parenteral route where delivery is by an injectable form, such as, for example, by a rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, intracavernosal, subcutaneous, transdermal or intramuscular) route.

By way of further example, the pharmaceutical composition of the present invention may be administered in accordance with a regimen of 1 to 10 times per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Disorders

The present invention is believed to have a wide therapeutic applicability—depending on inter alia the selection of the one or more NOIs.

For example, the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### DESCRIPTION OF THE DRAWINGS

Further preferred features and embodiments of the invention will now be further described only by way of non-limiting example with reference to the following Figures and accompanying Examples:

FIG. 1 is a representattion of pSFV.EIAV-lacZ;

FIG. 2 is a representation of SFV.synGag/pol;

FIG. 3 is a representation of SFV.VSV-G;

FIG. 4 is a schematic representation of the steps in the replication of the alphavirus genome;

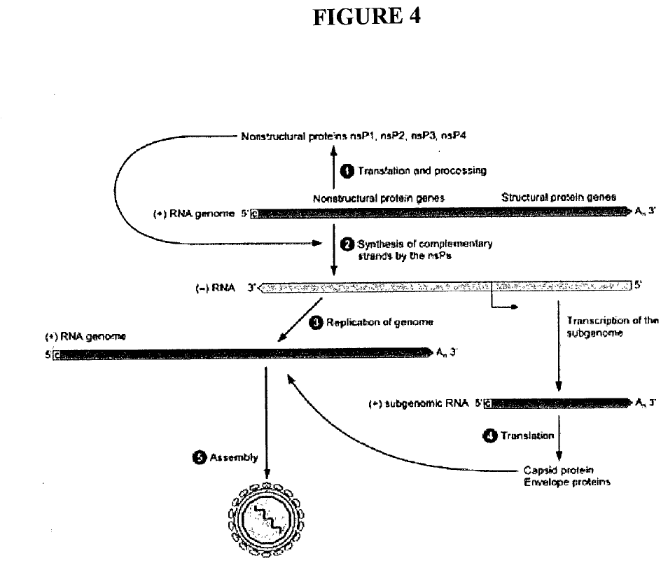

FIG. 5 is a graph showing the results of FACs analysis;

FIGS. 6A and B shows the results of Western blot analysis;

FIGS. 7A and B show the results of transduction of 293T cells;

FIG. 8 shows the results of RNA analysis.

### SUMMARY OF RESULTS

The use of alphaviruses for the cytoplasmic production of high titre retroviruses and lentiviruses has great potential for gene therapy applications. One advantage being the ability of genes containing introns and control/regulatory regions to be retained and packaged into retrovirus/lentivirus vectors. Normally introns are lost due to RNA processing in the nucleus of the retrovirus vector producer cell line. Introns have been shown to be beneficial in many ways in influencing gene expression. Li and Garoff have successfully produced transduction competent retrovirus (MLV) vectors to high titres (1×10⁶) from RNA expressed in the cytoplasm. This system worked well for generating recombinant MLV but not for producing recombinant EIAV where transduction levels were very poor. The EIAV structural proteins and enzymes were produced to high levels upon transfection of human embryonic kidney cells (293T) with the 3 RNA EIAV vector components using SFV. Functional reverse transcriptase was confirmed using PERT assays and Western blot analysis demonstrated that a high level of capsid protein p26 was being synthesised. RNA analysis indicated that amounts of genomic and subgenomic RNA were low. However RT PCR showed that there was sufficient RNA genome being packaged which should have corresponded to a biological titre of 1×10⁵ and should not be a limiting factor. FACS analysis demonstrated that the level of VSV-G in transfected 293T cells was also not limiting.

From our studies we have demonstrated that budding of EIAV vector particles does not occur in BHK-21 cells when transfected with SFV/EIAV RNA. Such a block in EIAV replication has not previously been reported. The absence of the EIAV capsid protein p26 in the culture medium suggests that the budding of extracellular vector has been blocked. The presence of substantial amounts of p26 in 293T cell culture medium demonstrates that EIAV vector budding occurs and is therefore not blocked as in BHK-21 cells. A different processing profile was also observed for gag proteins suggesting incorrect or incomplete gag processing within BHK cells. Due to this observed SFV/EIAV block in BHK-21cells, future studies with SFV/EIAV will have to utilize human cell lines such the embryionic kidney cell line 293T. This is surprising as the majority of SFV studies and indeed SFV/MLV chimeric studies have used the superior cell line BHK-21. (Wahfors et al, 1997; Li and Garoff, 1996). It has been reported that transfection efficiencies of up to 100% can be achieved using BHK cells. Li and Garoff demonstrated budding and achieved titres of 1×10⁶ for the production of recombinant MLV vector particles using the SFV vector system. (Li and Garoff, 1996; Li and Garoff, 1998). However, when the same strategy was used to generate recombinant EIAV via SFV, budding of EIAV was not observed. Similar findings may also be extrapolated to generating other lentiviruses such as HIV-1 in BHK cells via the SFV expression system. These findings would support a move away from using rodent models for lentiviral replication studies and has huge implications for HIV research.

### EXAMPLES

### Example 1

The construction of SFV/EIAV hybrid vectors.

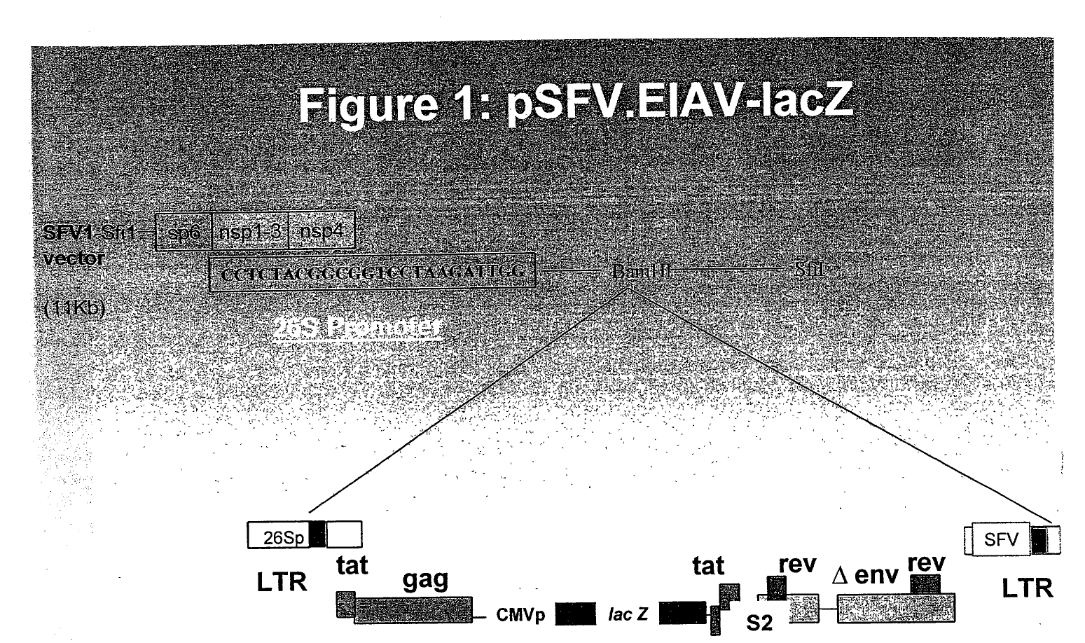

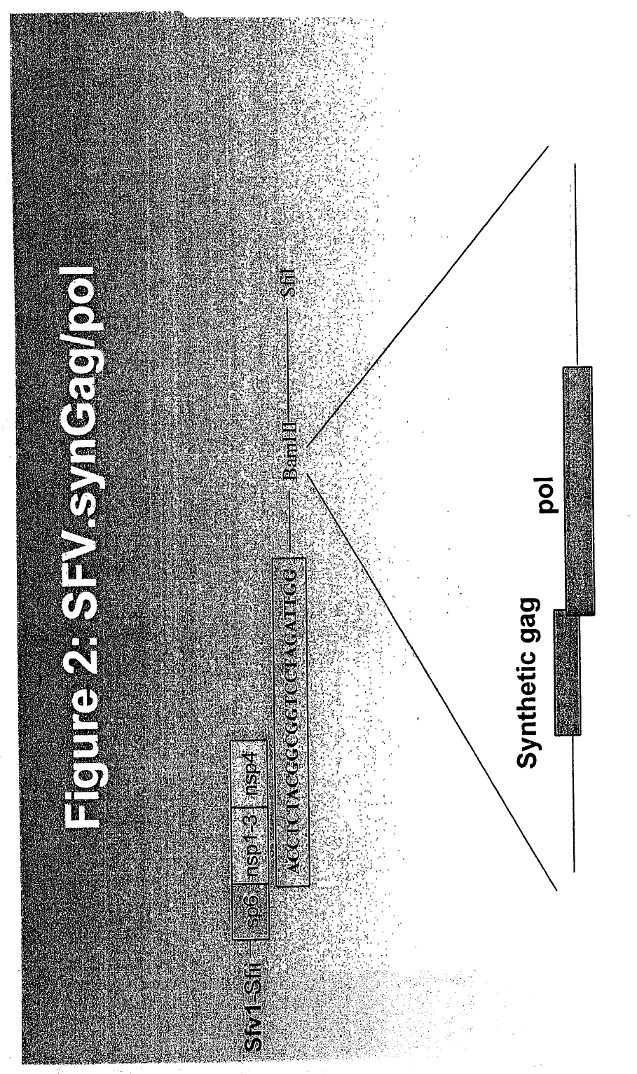

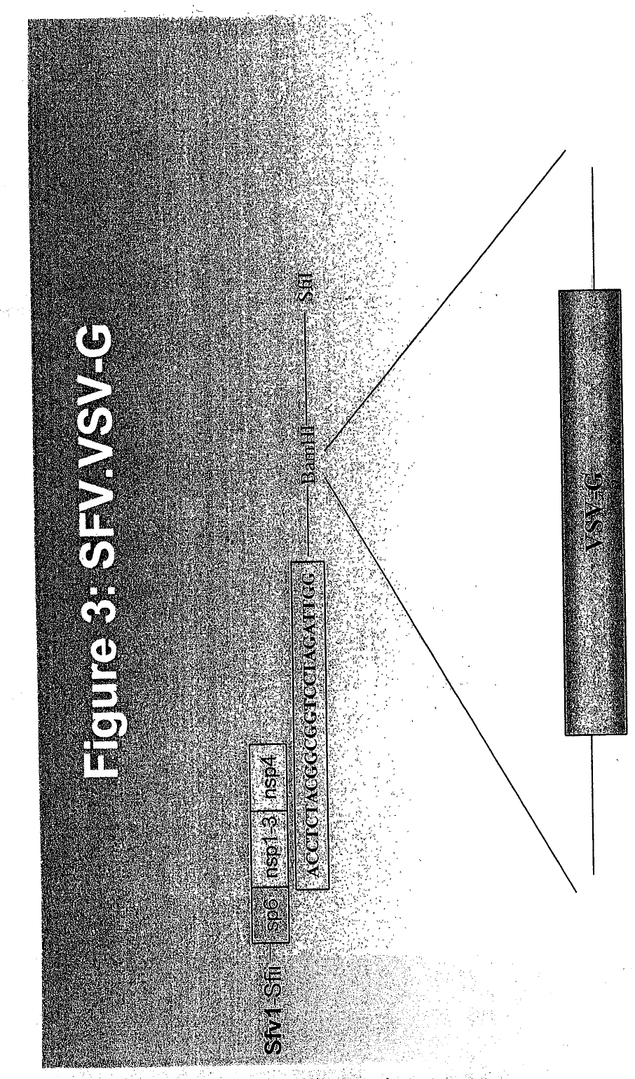

Plasmid construction: Replication incompetent EIAV particles were produced using Semliki Forest Virus derived RNA expression vectors. EIAV was expressed as three separate components, vector genome (pSFV.EIAV.lacZ—FIG. 1), gag/pol (pSFV.Esyn.gag/pol—FIG. 2) and envelope (pSFV.VSV-G—FIG. 3) to minimise any risk of producing replication competent viruses (RCRs). Viral particles were pseudotyped with VSV-G envelope glycoprotein (Burns et al, 1993). Each component was inserted downstream of the subgenomic 26S promoter of pSFV1-SfiI (Li and Garoff, 1996). A β-galactosidase gene driven by the HCMV IE enhancer/promoter was inserted internally in the construct pSFV.EIAV.lacZ as a reporter gene for transduction.

The plasmid SFV1 (Lijestrom and Garrof, 1991) was modified to contain a unique SfiI site to facilitate linearisation (SFVI-Sfi). The usual site for the linearisation of SFVI, SpeI could not be used as the EIAV genome also contained a SpeI site.

Construction of SFV-1.SfiI SFV38

Construction of SFV-1.SfiI

pSFV-1 contains a unique SpeI downstream of the SFV sequence which is used to linearise the plasmid prior to its use in in vitro transcription reactions. Since the EIAV vector components have SpeI sites the SpeI site in SFV-1 needed to be altered to be replaced with a restriction site not present in any of the EIAV vector system components. This alteration was made by inserting oligonuclotides corresponding to a SfiI site into the SpeI of SFV-1. The oligonucleotides were:

| | |
|---|---|
| SfiI POS: | CTAGTGGCCAGTGCGGCCACCTGA |
| SfiI NEG: | CTAGTCAGGTGGCCGCACTGGCC |

The presence of a unique SfiI site was confirmed by restriction digestion.

Insertion of SFV Sequences in the U3 Region of the EIAV 3′LTR

The sequences required for efficient transcription from the SFV subgenomic promoter extend into the transcript itself. For expression of a protein from a transcript made from this promoter, for example the EIAV gag/pol protein placed downstream of the SFV promoter, this characteristic is not a problem. However for production of a transcript representing a retroviral genome it does cause a difficulty. This arises because the retroviral vector transcript will have an 5′extension derived from SFV which after the ‘first strand jump’ step of reverse transcription will not have a complementary sequence (within the 3′LTR) to which to anneal. This lack of complementarity causes very inefficient negative strand DNA synthesis and poor vector titre. The solution is to alter the 3′LTR to match the 5′ end of the transcript. This necessitates insertion of 38 nucleotides SFV sequence immediately upstream of the R region of the EIAV 3′LTR.

This was achieved by the following manipulations: pONY4G (WO99/32646) was digested with BglII and BamHI and the 5996 bp band excised and religated. Following transformation into E.coli, XL-1 Blue, colonies were selected for the presence of this ligation product, termed pONY4G BgBam. This plasmid is the same as pONY4G except that all sequences from the 5′ end of the CMV promoter through to just upstream of the GFP reporter gene are deleted. This plasmid contains unique MluI and MunI sites in the 3′LTR region which allow insertion of oligonucleotides corresponding to the sequences of the subgenomic promoter of SFV which are present at the 5′end of the transcript made by the SFV subgenomic promoter. The oligonucleotides corresponding to the 38 nucleotides derived from the SFV subgenomic promoter (SFV38) were:

SFV38 POS:
CGCGTATTGGTGCGTTAATACACAGAATTCTGATTGGATCCC
SFV38 NEG:
AATTGGGATCCAATCAGAATTCTGTGTATTAACGCACCAATA

These were annealed and inserted into pONY4G BgBam digested with MluI and MunI. After ligation and transformation clones in which the SFV38 sequence were inserted were selected on the basis of restriction digestion and then a number of these clones were further analysed by sequencing the region containing the putative SFV38 insert. One plasmid named pONY4G BgBam SFV38 was selected for further work. The fused sequence of the SFV38-EIAV sequence present in this plasmid is designed to be identical to the sequence of transcript made by fusion of the SFV subgenomic promoter and 5′ end of the EIAV vector sequence as follows: pONY4 GBgBamSFV38 was cleaved with PvuII and Smal and the fragment containing SFV38 at the 3′LTR was inserted into the vector pSFV1.Sfi cleaved with Smal. This was then cleaved with NotI and NspV and the resulting fragment encoding the SFV38.3′LTR sequence was subcloned into the vector pSL1 180 also cleaved with NotI and NspV. PSL1180 bothLTRs were then cleaved with Smal and NspV and into this vector was inserted a MunI filled NspV fragment from pONY 2.11lacZ (WO99/32646) providing the 5′LTR. This was cleaved with NotI and NspV and the fragment ligated back into SFV3′LTR vector cleaved with NotI and NspV.

pSFV.VSV-G

An expression plasmid encoding the vesicular stomatitis virus G protein (VSV-G) was used to supply the envelope for peudotyping the viral particles. This is a favoured envelope for the pseudotyping of lentiviral core particles. The construct pSFV.VSV-G was produced by cleaving the plasmid pRV67 with Smal and altering this to a BamHI site using BamHI linkers (Stratagene). The VSV-G coding region was isolated as a BamHI fragment and inserted into the BamHI cloning site pSFV1-Sfi.

pEsynGag/polKosak-BamHI

The sequence of the gag-pol gene of EIAV had been (codon optimised) altered so that the codon-usage was that of a highly expressed mammalian gene. An expression plasmid encoding this sequence under the control of the SFV subgenomic promoter was constructed. The construct EsynGag/polKozak-BamHI was produced by cleaving the plasmid EsynGag/polKozak (PCT/GB01/01784) with EcoRI, performing a Klenow fillin reaction to produce blunt ends and inserting a BamHI site using BamHI linkers. This BamHI fragment encoding EsynGag/pol sequence was inserted into the BamHI cloning site of SFV1.Sfi downstream of the subgenomic promoter to produce pSFV.EsynGag/pol (PCT/GB01/01784).

pSFV.EIAV.lacZ

A genome expression plasmid was used encoding the genome, modified integration requirements (LTRs) and a reporter gene encoding α-galactosidase. The SFV recombinant genome (pSFV.EIAV.IacZ) extends from the SP6 promoter to the unique Sfi1 site. The construct contains, in 5′ to 3′ direction, (I) the 5′ replication signals of SFV RNA, (ii) genes encoding the SFV replication complex (non structural proteins, nspl-4), (iii) the internal subgenomic promoter of SFV, (iv) the recombinant EIAV genome including the 5′ R-U5, the encapsidation signal (Ψ), the LacZ gene and the modified 3′ U3/Rregion.

The construct pSFV.EIAV.lacZ was designed to contain a SFV specific 38 bp sequence (part of the subgenomic promoter) at the 3′ end of the genome to complement the 5‘end to produce identical LTR’s and therefore facilitate reverse transcription. Expression of the EIAV genome is regulated by the SFV sub-genomic promoter.

### Example 2

Reducing the EIAV Genome Construct.

To determine whether the actual size of the genome construct was a limiting factor in the generation of transduction competent EIAV vector particles via SFV, pSFV.EIAV.lacZ was reduced by 4 kb. As pSFV.EIAV.lacZ spans 18.7 kp, the size of the construct may be limiting the system at either the (in vitro) transcription stage or first strand synthesis. A smaller genome plasmid was constructed by replacing the lacZ gene from SFV.EIAV.lacZ with GFP from pONY 8.1 G (WO99/32646). SFV.EIAV.lacZ was cleaved with NspV and BbvCl to produce a reduced vector of 12.5 kb (and release the lacZ gene cassette and CMV promoter). A GFP gene cassette including the CMV promoter replaced lacZ by cleaving pONY8.1G with AccI and BbvCI and inserting the fragment encoding GFP (1.8 kb) into the SFV vector. (NspV and AccI produce complementary ends). The resulting plasmid SFV.8.1G has a smaller genome of only 14.3 kb. After reducing the vector genome by 4 kb, transduction competent recombinant EIAV particles were generated. However, initially only extremely low titres were achieved. This result would support the theory that the actual size of genome construct may detrimentally influence the efficiency of the chimeric system and by reducing the overall size of the vector allows integration.

### Example 3

RNA Transcription and Transfection:

As western blot analysis showed that incorrect processing had occurred in BHK cells, human 293T cells were used as an alternative. Electroporation of BHK cells has traditionally been the method of choice for SFV mRNA transfections. However, the cationic liposome DMRIE-C was used for 293T transfections as this method is commonly used for transfecting these cells. Each pSFV/EIAV construct was linearised with SfiI and used as a template for in vitro transcription using SP6 RNA polymerase (Ambion's Message Machine). 293 T cells (60-80% confluency) were transfected in 35 mm dishes with 6 μg of each purified full length synthetic SFV/EIAV RNA transcript using 10 μl of the cationic liposome reagent DMRIE-C in 1 ml of OPIMEM (Ciccarone et al, 1994). At 5 hours post transfection, the monolayer was washed with a further 1 ml of OPTIMEM replaced with 2 ml of medium supplemented with 10%FCS. At approximately 20 hours post transfection the cell culture medium containing the virus was harvested and filtered (0.45 μm filter).

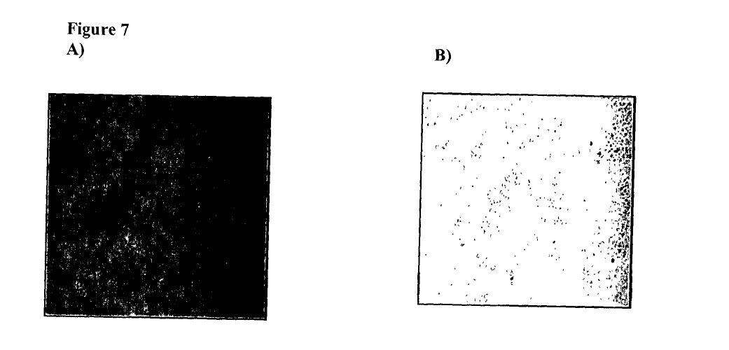

In relation to FIG. 7, in order to determine the transfection efficiency of 293Ts via DMRIE-C, 293T cells were transfected with pSFV.lacZ RNA (5-10 μg). At 20 hours post transfection the monolayers were reacted with X-gal. SFV.lacZ RNA transfected (A) and mock-transfected (B) cells are shown. Approximately 30-40% of cells appeared to be transfected (blue).

Between 39-40% of pSFV-lacZ RNA transfected cells stained blue with X-gal. FACS analysis reported 36% (VSV-G) and 44% (peudotyped with human CMV VSV-G) of cells were expressing VSV-G. Transfection efficiecies of up to 44% were achieved and will be improved by further optimisation of this system. Western blot analysis demonstrated that EIAV gag/pol proteins were correctly processed and budded from transfected human 293T cells. BHK (the major cell type used for SFV studies) however showed inefficient gag processing and no budded virus.

Western Blot Analysis

In order to determine the level of gag expression, processing and budding, western blot analysis was performed on the cell extracts and supernatants. The cells were washed once in PBS and centrifuged at 500 g for 1 min and resuspended into PBS, 0.1%NP40. Protein content was measured by Bradford assays and 15 μg of each protein sample was analysed on 10% PAGE gels. Culture medium was also analysed by PAGE.

Western blot analysis demonstrated that in BHK cells, budding did not occur as the p26 capsid protein was not detected in the culture medium of transfected BHK cells.

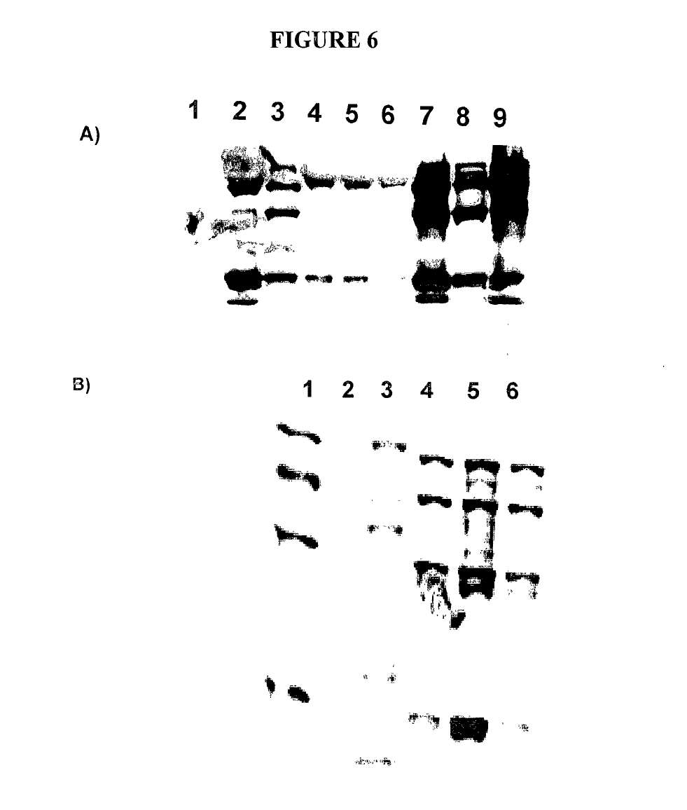

In FIGS. 6A—293T cells transfected with 5-10 μg (pSFV.Esyngag/pol) RNA produced gag proteins including precursor and cleavage products with significant amounts of the p26 capsid protein that is predominantly recognised by the polyclonal horse serum used. A) gag/pol proteins were detected in cell supernatants (lanes 4-6) and in cell lysates (lanes 7-9). EIAV+ve controls for cell lysate (cell lysates from a transient EIAV viral prep) (lane 3), supernatant (lane 2) and mock supernatant (lane 1) were included.

In FIG. 6B—Shows lysates from cells transfected with SFV.Esyn.gag/pol RNA transcripts: Lanes 4-6 show 293T cells (5-10 g). Incomplete gag/pol processing in BHK cells is observed (lane 3) and EIAV+ve control (lane 1). Lane 3 shows BHK cell lysates transfected with all three RNA transcripts. This would suggest incomplete processing of gag/pol proteins. Additionally, there was no budded virus detected when the cell supernatants were analysed by western blotting. These observations suggest that EIAV replication may be blocked in certain rodent cell lines.

Western blotting analysis therefore demonstrated that in BHK cells, budding did not occur as the p26 capsid protein was not detected in the culture medium of transfected BHK cells. Additionally, the profile of gag proteins from the BHK cell lysates differered greatly from that analysed from the 293T cells. Incomplete processing of the Gag/pol proteins could occur in BHK cells which may correlate to reported findings of blocks in HIV replication in many rodent cell lines (Bieniasz and Cullen, 2000). Such findings have so far not been reported for the replication of EIAV. Additionally, an insect cell-specific defect in simian D-type retroviral particle assembly has recently been reported, as has a defect in HIV-1 assembly in mouse NIH 3T3 cell (Parker and Hunter, 2000; Mariani et al, 2000). Host cell lines could have specific defects in the late stages of the retroviral/lentiviral life cycle, implicating the involvement of cellular factors during Gag processing, transport, viral assembly and budding.

FACS Analysis

FACS analysis was carried to check whether enough envelope was being produced by the subgenomic promoter of SFV.VSV-G and also as a indication of transfection efficiency. 293T cells transfected with 6 μg of each mRNA transcript using DMRIE-C were harvested at 20 hours post transfection. 1×10⁵ transfected cells were used for FACS analysis. To half of the cells, the monoclonal antibody (Mouse monoclonal antibody, clone P5D4, to a peptide from the vesicular stomatitis virus glycoprotein conjugated to HRP, Boehringer Mannheim) was added at a dilution of 1:2 in PGB (20 mM glucose, 1%BSA in PBS). Cells were incubated for 30 min room temperature and then washed in PGB. Secondary antibody was added to these cells and to the initial second half of cells at a dilution of 1:10 (DAKO) for 30 min at room temperature.

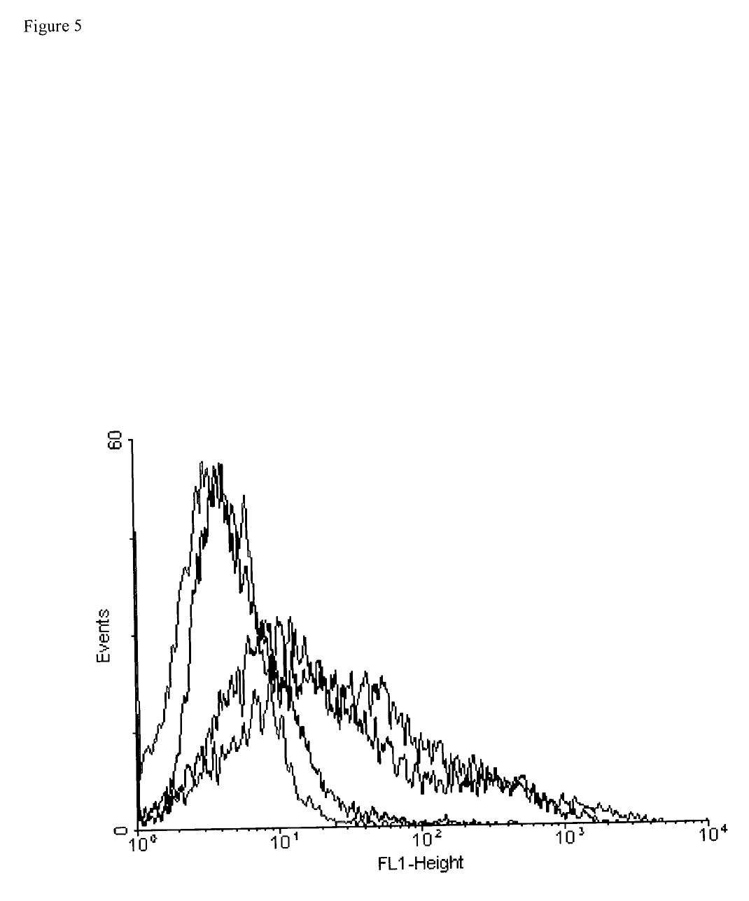

With regard to FIG. 5, transfected 293T cells were harvested at 20 hpi and analysed by FACS to measure levels of VSV-G expression. Cells with secondary alone acted as a control for non specific binding of secondary to cells not expressing VSV-G. Dead cells were stained with ToPro 3 (Molecular Probes) and were excluded from the final analysis. The transfection efficiency as measured by FACS analysis was between 36-44% fluorescent cells indicating expression of VSV-G.

As shown in FIG. 5 black lines indicate EV11E producer cells expressing VSV-G. Blue indicates the transfected cells expressing VSV-G (red and green show live cells with secondary antibody only). The results show that VSV-G expression level is greater than the secondary only control and is slightly less that the EV 11 producer which yields biological titres between 10-3 and 10-4. These results suggest that VSV-G expression is not a limiting factor and that biological titres of SFV.EIAV virus should lie within 10-2 and 10-3.

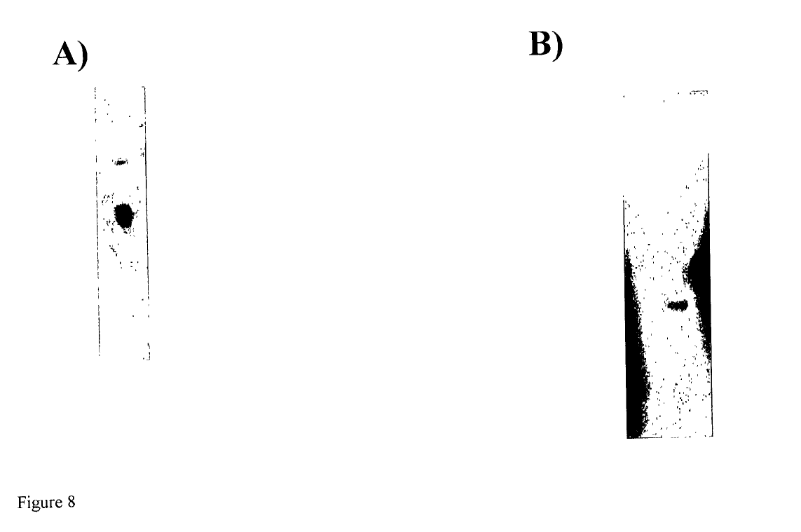

RNA Analysis (FIG. 8)

In order to determine whether the subgenomic RNA encoding the EIAV genome was being produced, Northern blotting analysis was performed. Transfected 293T cells were harvested at 18 hours post transfection. RNA was isolated using a Total RNA kit (Quiagen). The RNA was separated on a formamide gel and immobilized onto a Hybond-N+ nylon membrane (Amersham Life Science) by blotting. The transferred RNA was fixed to the membrane by UV crosslinking A DNA fragment encoding lacZ was labelled using a Random Primer Labelling kit (Stratagene) and used to probe the membrane. Plate A) indicates the SFV3lacZ+ve control (the same lane which is over expressed in plate B). Also in plate B) the two lanes with genomic (upper band) and subgenomic RNA (lower band) are clearly visible.

The results from the RNA analysis would suggest that the levels of RNA are low but that both genomic and sub-genomic species were present. This result demonstrates that the sub-genomic promoter is active in the expression of the EIAV genome.

### Example 4

Validation of SFV/EIAV with SFV/MLV.

Since titres of 1×10⁶ were achieved by Li and Garoff's lab for the SFV/MLV chimeric system further experiments were set up to determine why such low titres were achieved for the SFV/EIAV system. The SFVMLV constructs described in the original patent (WO9815636): SFV1/LN3i, SFV1/MLVgag/pol, SFVPr80env (or SFV-VSV-G) were used in experiments to validate the SFV1/EIAV system.

Transfections were set up with combinations of the following RNA run off transcripts:

SFV1/LN3i, SFV1/MLVgag/pol, SFVPr80env

SFV1/LN3i, SFV1/MLVgag/pol, SFV/VSV-G

SFV.EIAV.lacZ, SFVEsyn.gag/pol, SFV/VSV-G

SFV.8.1G, SFVEsyngag/pol, SFV/VSV-G

Cells from the (3 RNA cassette) transfections were assayed using FACS for VSV-G expression (transfection efficiency). RT PCR using real time PCR (Taq-man) was used to quantify the abundance of packaged viral genomes. PERT assays were performed to determine the levels of RT activity.

FACS analysis indicated that for SFVEIAV, 13.36% of cells were transfected. The reduced genome gave a value of 18.24% and the SFVMLV cells gave a value of 40.48%. The +ve control for EIAV gave a value of 69.76%.

Transduction of D17 Cells

Actual transduction was assessd by innoculating D17 cells with the transient SFV.EIAV viruses: SFV.EIAV.lacZ and SFV.8.1G and monitoting for either blue colony forming units for the former and fluorescence for the latter. D17 cells were seeded at 8×10⁴ and transduced with neat, 10⁻² and 10⁻³ viral dilutions. 500 μl of each dilution (in complete medium plus polybrene) was used to innoculate D17 cells for 4 hours, after which 1 ml of complete medium was added. Each plate was incubated for 3 days at 37° C. At 3 days post transduction, each virus titre was calculated using their respective 2 reporter genes.

SFVEIAV (lacZ)

SFV8.1G (GFP)

SFVLN3i (Neo)

For Neo selection: The transduced cells were plated onto dishes and the cuture medium containing G418 (1 mg/ml) was added. The selective medium was replaced again after 48 hrs. Isolated neo resistant clones were counted after 8 days. The majority of cells died after the addition of G418 at 1 mg/ml.

For LacZ selection, transduced cells were fixed with 4% neutral buffered formalin and stained with X-Gal for several hours/overnight. Blue colony forming units were not observed in any of the test samples.

For GFP selection, the culture medium was replaced and the cells transferred to 32° C. Fluorescent cells were counted using a fluorescent microscope after a further 2 days. A titre of 2 fluorescent colonies per ml was achieved from transduced D17cells after 48 hours.

PERT and RT PCR data (Table 1)

Viral vector RNA was isolated from culture medium and treated with DNAse I. RNA was quantified using Real Time PCR (Taqman) performed on an ABI Prism 7700 Sequence Detection System. For SFV.EIAV RNA quantification the CMV sequence was amplified and for SFV.MLV, the Noemycin resistance gene was amplified. Reverse trancriptase activity was also measured using this machine (PERT-Fluorescent Product Enhanced Reverse Transcriptase).

| CMV | Minus Rt | Plus Rt | NEO | Minus Rt | Plus Rt |
|---|---|---|---|---|---|
| EiavStd-2 | 29 | 26 | MLVStd-2 | 35.9 | 26.4 |
| Sfveiav-2 | 34 | 28.1 | SfvMlv-2 | 35.6 | 25.6 |
| EiavStd-1 | 25.4 | 22.4 | MLVStd-1 | 37.3 | 23.1 |
| Sfveiav-1 | 34.4 | 24.1 | SfvMlv-1 | 35.1 | 23.4 |
| PERT | 24 hpi | 48 hpi | | | |
| EiavStd-2 | 21.1 | | | | |
| Sfveiav-2 | 26.7 | 23.9 | | | |
| SfvMLV | 31.6 | 28.7 | | | |

Reverse transcriptase activity for both SFV.EIAV and SFV.MLV was highest at 48hpi (lowest Ct values). Both systems demonstrated functional RT activity and SFV.EIAV produced greater activity than the SFV.MLV. Reverse transcriptase activity is therefore not a limiting factor in the SFV.EIAV vector system. PCR amlplification was achieved for each of the target sequences (Neo/MLV and CMV/EIAV) demonstrating that each gemone was present in relatively high amounts in comparison to each of the standard controls. This demonstrared that the sub genomic promoter for SFV.EIAV is functional generating full sub genomic RNA encoding the EIAV vector genome. Since both RNA standards originated from viruses with biological titres of 1×10⁶, the SFV.EIAV vector particles would therefore be expected to yield a biological titre of at least 1×10⁵.

### REFERENCES

All references referred to below and also above are herein incorporated by reference.

1. Li, K and Garoff, H. (1996). Proc Natl Acad Sci USA. 93:11658-11663.

2. Lever, A. M. L. (1996). Gene Therapy. 3:470-471.

3. Mitrophanous, K et al., (1999). Gene Therapy. 6:1808-1818. Liljestrom, P and Garoff, H. (1991). Biotechnology. 9:1356-1361.

5. Bums, J C et al., (1993). Proc Natl Acad Sci USA. 90:8033-8037.

6. Ciccarone, V et al., (1994). FOCUS. 16: 94.

7. Mariana, R et al., (2000). Journal of Virology. 74:3859-3870.

8. Bieniasz, P and Cullen,B (2000). Journal of Virology. 74:9868-9877.

9. Parker, S. D and Hunter, E. (2000) Journal of Virology. 74 784-795.

10.Wahlfors, et al, (1997), Human gene therapy. 8; 2031-2041.

11. Li and Garoff, (1998) Proc Natl Acad Sci USA. 95, 3650-3654.

1. An alpha-lentivirus vector comprising at least one alphaviral component and at least one lentiviral component, wherein the lentiviral component is capable of being packaged into a lentiviral particle after introduction of said vector into a human cell.

2. A vector according to claim 1 wherein the vector is a alphavirus expression system comprising at least one lentiviral component, wherein the lentiviral component is capable of being packaged into a lentiviral particle after introduction of said vector into a human cell.

3. A vector according to claim 1 or claim 2 wherein the lentiviral component corresponds to a lentiviral genome.

4. A vector according to claim 3 wherein the lentiviral component comprises an exogenous nucleotide sequence of interest (NOI).

5. A vector according to any preceding claim wherein the lentiviral component is derived from a lentivirus selected from the group consisting of HIV, SIV, VMV, CAEV, EIAV, FIV and BIV.

6. A vector according to any preceding claim wherein the alphaviral component is derived from an alphavirus selected from the group consisting of Semliki Forest virus (SFV), Sindbis virus, Ross River virus and Venezuelan, Western and Eastern Equine Encephalitis viruses.

7. A vector according to any one of the preceding claims wherein the composition comprises an RNA transcription start site for the lentiviral vector, and wherein the nucleotide sequence encoding the lentiviral component is operably linked to a promoter comprising an upstream promoter component located upstream of the RNA transcription start site and a downstream promoter component located downstream of the RNA transcription start site.

8. A vector according to claim 7 wherein the downstream promoter component is upstream of the polynucleotide sequence encoding the lentiviral vector.

9. A vector according to claim 7 or claim 8 wherein the promoter is an alphavirus promoter.

10. An alphavirus particle which contains an alphavirus-lentivirus vector of any preceding claim.

11. A lentiviral particle obtainable from the expression in a human cell of the alpha-lentivirus vector of any on of claims 1 to 9.

12. A human cell that contains an alpha-lentiviral vector of any one of claims 1 to 9.

13. A human cell according to claim 12 wherein the cell is selected from the group consisting of a HEK 293, HEK 293T, TE 671 and HT 1080 cell.

14. A method of producing lentiviral particles comprising infection a human cells with an alpha-lentivirus vector of any one of claim 1 to 9 optionally together with other vectors specifying the production of lentiviral particles, incubating the cells, and collecting the lentiviral particles.

15. A method of transducing cells comprising the steps and preparing lentivirus particles according to the method of claim 14 and using the lentivirus particles to infect cells.

16. A method of transducing cells comprising using the lentivirus particle of claim 11 to infect cells.
